(19) United States
(12) Patent Application Publication
(10) Pub. No.: US 2025/0109158 A1
(43) Pub. Date: Apr. 3, 2025

(54) METHOD OF MANUFACTURE OF PENTAAZA MACROCYCLIC RING COMPLEX, INTERMEDIATES, AND PRODUCTS THEREOF

(71) Applicant: Galera Labs, LLC, Malvern, PA (US)

(72) Inventors: Jeffrey A. SCHOLTEN, Chesterfield, MO (US); Otto F. SCHALL, Creve Coeur, MO (US); Dennis P. RILEY, Chesterfield, MO (US); Robert A. BEARDSLEY, University City, MO (US)

(21) Appl. No.: 18/598,300

(22) Filed: Mar. 7, 2024

Related U.S. Application Data
(60) Provisional application No. 63/451,116, filed on Mar. 9, 2023.

(51) Int. Cl. C07F 13/00

(52) U.S. Cl. CPC C07F 13/005

(57) ABSTRACT

A process of preparing a pentaaza macrocyclic ring complex is provided, the process comprising, in a cyclization stage, reacting a tetraamine compound or salt thereof with a diacetyl pyridine compound, and a source of manganese (II) ion, in the presence of a tertiary amine base to form a bisimine compound, and in a reduction stage performed after at least 50% by weight of the diacetyl pyridine compound has reacted in the cyclization stage, performing a catalytic hydrogenation reduction reaction on the bisimine compound to form the pentaaza macrocyclic ring complex having at least 99% optical purity. A pentaaza macrocylic ring complex produced by such process is also provided, the pentaaza macrocyclic ring complex having at least 99% optical purity without requiring further optical purification.

### CROSS-REFERENCE TO RELATED APPLICATIONS

The present application claims benefit of priority to U.S. Provisional Patent Application No. 63/451,116, filed Mar. 9, 2023, the entire content of which is hereby incorporated by reference herein in its entirety, as if recited in full herein.

The present disclosure generally relates to a method of manufacture of pentaaza macrocyclic ring complexes, as well as the intermediates and final products produced thereby.

Manganese-containing pentaaza macrocyclic ring complexes having the macrocyclic ring system corresponding to Formula A have been shown to be effective in a number of animal and cell models of human disease, as well as in treatment of conditions afflicting human patients.

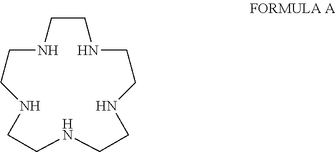 [SMILES: C1CNCCNCCNCCNCCN1]

For example, in a rodent model of colitis, one such compound, GC4403, has been reported to very significantly reduce the injury to the colon of rats subjected to an experimental model of colitis (see Cuzzocrea et al., Europ. J. Pharmacol., 432, 79-89 (2001)).

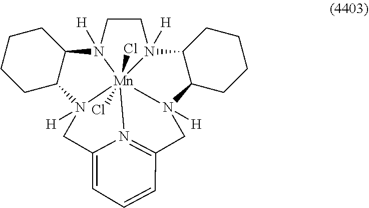

GC4403 has also been reported to attenuate the radiation damage arising both in a clinically relevant hamster model of acute, radiation-induced oral mucositis (Murphy et al., Clin. Can. Res., 14(13), 4292 (2008)), and lethal total body irradiation of adult mice (Thompson et al., Free Radical Res., 44(5), 529-40 (2010)). Similarly, another such compound, GC4419, has been shown to attenuate VEGFr inhibitor-induced pulmonary disease in a rat model (Tuder, et al., Am. J. Respir. Cell Mol. Biol., 29, 88-97 (2003)). Additionally, another such compound, GC4401 has been shown to provide protective effects in animal models of septic shock (S. Cuzzocrea, et. al., Crit. Care Med., 32(1), 157 (2004) and pancreatitis (S. Cuzzocrea, et al., Shock, 22(3), 254-61 (2004)).

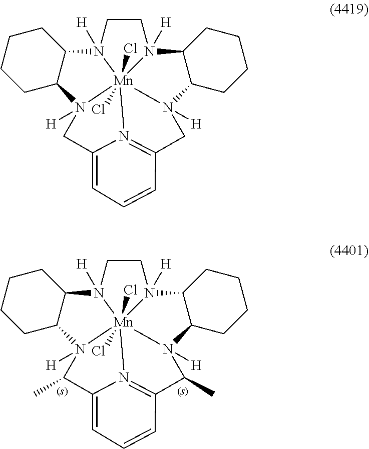

Certain of these compounds have also been shown to possess potent anti-inflammatory activity and prevent oxidative damage in vivo. For example, GC4403 has been reported to inhibit inflammation in a rat model of inflammation (Salvemini, et. al., Science, 286, 304 (1999)), and prevent joint disease in a rat model of collagen-induced arthritis (Salvemini et al., Arthritis & Rheumatism, 44(12), 2009-2021 (2001)). Yet others of these compounds, MdPAM and MnBAM, have shown in vivo activity in the inhibition of colonic tissue injury and neutrophil accumulation into colonic tissue (Weiss et al., The Journal of Biological Chemistry, 271(42), 26149-26156 (1996)). In addition, these compounds have been reported to possess analgesic activity and to reduce inflammation and edema in the rat-paw carrageenan hyperalgesia model, see, e.g., U.S. Pat. No. 6,180,620.

Compounds of this class have also been shown to be safe and effective in the prevention and treatment of disease in human subjects. For example, GC4419 has been shown to reduce oral mucositis in head-and-neck cancer patients undergoing chemoradiation therapy (Anderson, C., Phase 1 Trial of Superoxide Dismutase (SOD) Mimetic GC4419 to Reduce Chemoradiotherapy (CRT)-Induced Mucositis (OM) in Patients (pts) with Mouth or Oropharyngeal Carcinoma (OCC), Oral Mucositis Research Workshop, MASCC/ISOO Annual Meeting on Supportive Care in Cancer, Copenhagen, Denmark (Jun. 25, 2015); Anderson, C., Phase 1b/2a Trial of Superoxide Dismutase Mimetic GC4419 to Reduce Chemoradiotherapy-Induced Oral Mucositis in Patients with Oral Cavity or Oropharyngeal Carcinoma, Int. J. of Radiation Oncol. Biol. Phys., Vol. 100, No. 2, pages 427-435 (2018)).

In addition, transition metal-containing pentaaza macrocyclic ring complexes corresponding to this class have shown efficacy in the treatment of various cancers. For example, certain compounds corresponding to this class have been provided in combination with agents such as paclitaxel and gemcitabine to enhance cancer therapies, such as in the treatment of colorectal cancer and lung cancer (non-small cell lung cancer) (see, e.g., U.S. Pat. No. 9,198,893) The 4403 compound above has also been used for treatment in in vivo models of Meth A spindle cell squamous carcinoma and RENCA renal carcinoma (Samlowski et al., Nature Medicine, 9(6), 750-755 (2003), and has also been used for treatment in in vivo models of spindle-cell squamous carcinoma metastasis (Samlowski et al., Madame Curie Bioscience Database (Internet), 230-249 (2006)). The 4419 compound above has also been used in combination with cancer therapies, such as in combination with a therapy involving administration of cisplatin and radiation, to enhance treatment in in vitro models (Mohanty et al., Proceedings: AACR Annual Meeting 2018, April 14-18 (2018)). (See also Sishc et al., Sci. Transl. Med., 2021; 13:1-13; Mapuskar et al., Cancer Res, 2017; 77(18): 5054-5067; Anderson et al. J. Clin Oncol., 2019; 37:3256-3265, Phase IIb, Randomized, Double-Blind Trial of GC4419 versus Placebo to Reduce Severe Oral Mucositis due to Concurrent Radiotherapy and Cisplatin for Head and Neck Cancer. Yet another pentaaza macrocylic ring complex that shows promise for therapeutic treatment is the compound GC4711, as described in WO 2017/027728 published on Feb. 16, 2017, which is hereby incorporated by reference herein in its entirety.

Accordingly, a need remains for enhanced methods of manufacture for pentaaza macrocyclic ring complexes such as GC4419, GC4711 and similar complexes, including via synthetic routes that provide for excellent yield and purity of the resulting complexes, as well as the intermediates and products themselves produced by such methods of manufacture.

Briefly, therefore, aspects of the present disclosure are directed to methods of manufacture of pentaaza macrocyclic ring complexes.

According to certain embodiments, aspects of the present disclosure are directed to process of preparing a pentaaza macrocyclic ring complex of Formula (I)(a) or (I)(b) below:

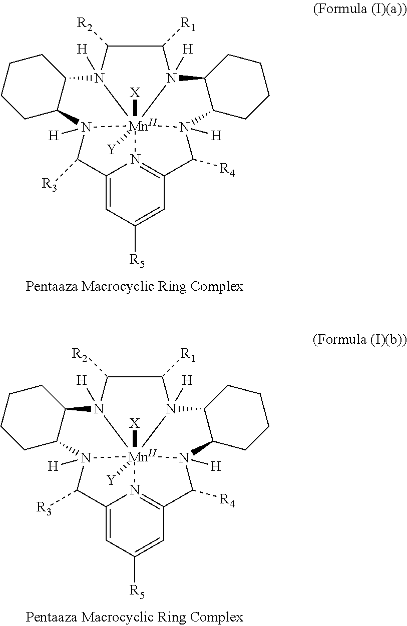

wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
the process comprising,
(A) in a cyclization stage, reacting a tetraamine product comprising a tetraamine compound of Formula (III)(a)(i) or Formula (III)(a)(ii) below, or a salt thereof, with a diacetyl pyridine compound of Formula (III)(b) below, and a source of manganese (II) ion corresponding to the formula Mn(X)(Y), in the presence of a tertiary amine base, wherein the tetraamine product comprises at least 99% optical purity of the compound of Formula (III)(a)(i) or Formula (III)(a)(ii), or salt thereof;

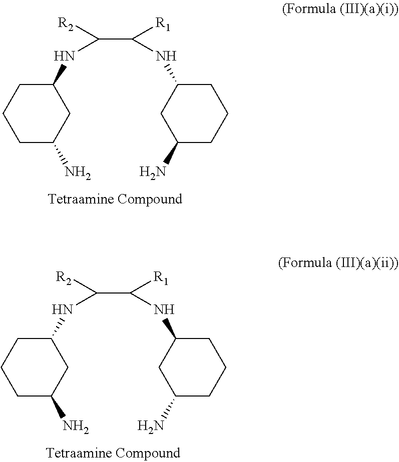

wherein R₁-R₄ are independently selected from group consisting of hydrogen and substituted or unsubstituted alkyl,

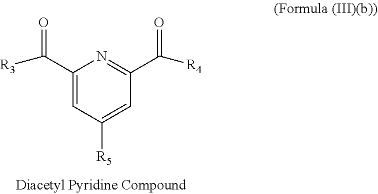

wherein R₃ and R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
to provide a cyclization stage product comprising a bisimine compound of Formula (II)(a) or (II)(b) below having at least 95% optical purity

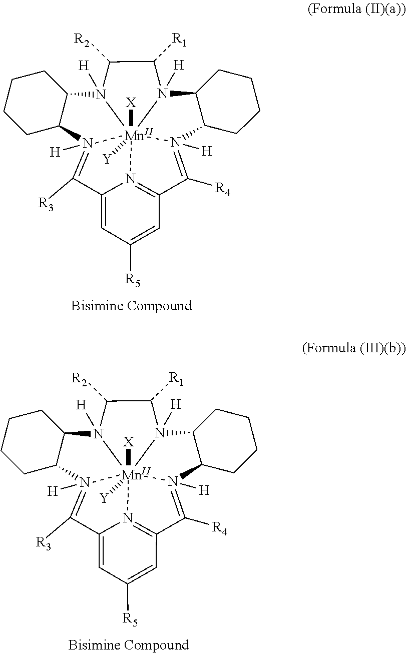

wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂, and
(B) in a reduction stage performed after at least 50% by weight of the diacetyl pyridine compound of Formula (III)(b) has reacted in the cyclization stage, performing a catalytic hydrogenation reduction reaction on the bisimine compound of Formula (II)(a) or Formula (II)(b) to form a reduction stage product comprising the pentaaza macrocyclic ring complex of Formula (I)(a) or Formula (I)(b) having at least 99% optical purity.

Aspects of the disclosure are further directed to a method of treatment of a condition in a patient, by administering compositions containing the pentaaza macrocyclic ring complex manufactured according to embodiments described herein.

Other objects and features of aspects of the disclosure are described hereinafter.

### Abbreviations and Definitions

The following definitions and methods are provided to better define the present invention and to guide those of ordinary skill in the art in the practice of the present invention. Unless otherwise noted, terms are to be understood according to conventional usage by those of ordinary skill in the relevant art.

“Acyl” means a —COR moiety where R is alkyl, haloalkyl, optionally substituted aryl, or optionally substituted heteroaryl as defined herein, e.g., acetyl, trifluoroacetyl, benzoyl, and the like.

“Acyloxy” means a —OCOR moiety where R is alkyl, haloalkyl, optionally substituted aryl, or optionally substituted heteroaryl as defined herein, e.g., acetyl, trifluoroacetyl, benzoyl, and the like.

“Alkoxy” means a —OR moiety where R is alkyl as defined above, e.g., methoxy, ethoxy, propoxy, or 2-propoxy, n-, iso-, or tert-butoxy, and the like.

“Alkyl” means a linear saturated monovalent hydrocarbon moiety such as of one to six carbon atoms, or a branched saturated monovalent hydrocarbon moiety, such as of three to six carbon atoms, e.g., C₁-C₆ alkyl groups such as methyl, ethyl, propyl, 2-propyl, butyl (including all isomeric forms), pentyl (including all isomeric forms), and the like.

Moreover, unless otherwise indicated, the term “alkyl” as used herein is intended to include both “unsubstituted alkyls” and “substituted alkyls,” the latter of which refers to alkyl moieties having substituents replacing a hydrogen on one or more carbons of the hydrocarbon backbone. Indeed, unless otherwise indicated, all groups recited herein are intended to include both substituted and unsubstituted options.

The term “C_{x-y}” when used in conjunction with a chemical moiety, such as alkyl and aralkyl, is meant to include groups that contain from x to y carbons in the chain. For example, the term C_{x-y} alkyl refers to substituted or unsubstituted saturated hydrocarbon groups, including straight chain alkyl and branched chain alkyl groups that contain from x to y carbon atoms in the chain.

“Alkylene” means a linear saturated divalent hydrocarbon moiety, such as of one to six carbon atoms, or a branched saturated divalent hydrocarbon moiety, such as of three to six carbon atoms, unless otherwise stated, e.g., methylene, ethylene, propylene, 1-methylpropylene, 2-methylpropylene, butylene, pentylene, and the like.

“Alkenyl” a linear unsaturated monovalent hydrocarbon moiety, such as of two to six carbon atoms, or a branched saturated monovalent hydrocarbon moiety, such as of three to six carbon atoms, e.g., ethenyl (vinyl), propenyl, 2-propenyl, butenyl (including all isomeric forms), pentenyl (including all isomeric forms), and the like.

“Alkaryl” means a monovalent moiety derived from an aryl moiety by replacing one or more hydrogen atoms with an alkyl group.

“Alkenylcycloalkenyl” means a monovalent moiety derived from an alkenyl moiety by replacing one or more hydrogen atoms with a cycloalkenyl group.

“Alkenylcycloalkyl” means a monovalent moiety derived from a cycloalkyl moiety by replacing one or more hydrogen atoms with an alkenyl group.

“Alkylcycloalkenyl” means a monovalent moiety derived from a cycloalkenyl moiety by replacing one or more hydrogen atoms with an alkyl group.

“Alkylcycloalkyl” means a monovalent moiety derived from a cycloalkyl moiety by replacing one or more hydrogen atoms with an alkyl group.

“Alkynyl” means a linear unsaturated monovalent hydrocarbon moiety, such of two to six carbon atoms, or a branched saturated monovalent hydrocarbon moiety, such as of three to six carbon atoms, e.g., ethynyl, propynyl, butynyl, isobutynyl, hexynyl, and the like.

“Alkoxy” means a monovalent moiety derived from an alkyl moiety by replacing one or more hydrogen atoms with a hydroxy group.

“Amino” means a —NR^{a}R^{b} group where R^{a} and R^{b} are independently hydrogen, alkyl or aryl.

“Aralkyl” means a monovalent moiety derived from an alkyl moiety by replacing one or more hydrogen atoms with an aryl group.

“Aryl” means a monovalent monocyclic or bicyclic aromatic hydrocarbon moiety of 6 to 10 ring atoms e.g., phenyl or naphthyl.

“Cycle” means a carbocyclic saturated monovalent hydrocarbon moiety of three to ten carbon atoms.

“Cycloalkyl” means a cyclic saturated monovalent hydrocarbon moiety of three to ten carbon atoms, e.g., cyclopropyl, cyclobutyl, cyclopentyl, or cyclohexyl, and the like.

“Cycloalkylalkyl” means a monovalent moiety derived from an alkyl moiety by replacing one or more hydrogen atoms with a cycloalkyl group, e.g., cyclopropylmethyl, cyclobutylmethyl, cyclopentylethyl, or cyclohexylethyl, and the like.

“Cycloalkylcycloalkyl” means a monovalent moiety derived from a cycloalkyl moiety by replacing one or more hydrogen atoms with a cycloalkyl group.

“Cycloalkenyl” means a cyclic monounsaturated monovalent hydrocarbon moiety of three to ten carbon atoms, e.g., cyclopropenyl, cyclobutenyl, cyclopentenyl, or cyclohexenyl, and the like.

“Cycloalkenylalkyl” means a monovalent moiety derived from an alkyl moiety by replacing one or more hydrogen atoms with a cycloalkenyl group, e.g., cyclopropenylmethyl, cyclobutenylmethyl, cyclopentenylethyl, or cyclohexenylethyl, and the like.

“Ether” means a monovalent moiety derived from an alkyl moiety by replacing one or more hydrogen atoms with an alkoxy group.

“Halo” means fluoro, chloro, bromo, or iodo, preferably fluoro or chloro.

“Heterocycle” or “heterocyclyl” means a saturated or unsaturated monovalent monocyclic group of 4 to 8 ring atoms in which one or two ring atoms are heteroatom selected from N, O, or S(O)n, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this Application as “bicyclic heterocyclyl” ring. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group is a saturated ring and is not fused to aryl or heteroaryl ring as stated above, it is also referred to herein as saturated monocyclic heterocyclyl.

“Heteroaryl” means a monovalent monocyclic or bicyclic aromatic moiety of 5 to 10 ring atoms where one or more, preferably one, two, or three, ring atoms are heteroatom selected from N, O, or S, the remaining ring atoms being carbon. Representative examples include, but are not limited to, pyrrolyl, pyrazolyl, thienyl, thiazolyl, imidazolyl, furanyl, indolyl, isoindolyl, oxazolyl, isoxazolyl, benzothiazolyl, benzoxazolyl, benzimidazolyl, quinolinyl, isoquinolinyl, pyridinyl, pyrimidinyl, pyrazinyl, pyridazinyl, triazolyl, tetrazolyl, and the like.

“Nitro” means —NO₂.

“Organosulfur” means a monovalent moiety a —SR group where R is hydrogen, alkyl or aryl.

“Substituted alkyl,” “substituted cycle,” “substituted phenyl,” “substituted aryl,” “substituted heterocycle,” and “substituted nitrogen heterocycles” means an alkyl, cycle, aryl, phenyl, heterocycle or nitrogen-containing heterocycle, respectively, optionally substituted with one, two, or three substituents, such as those independently selected from alkyl, alkoxy, alkoxyalkyl, halo, hydroxy, hydroxyalkyl, or organosulfur. Generally, the term “substituted” includes groups that are substituted with any one or more of C₁₋₄alkyl, C₂₋₄alkenyl, halogen, alcohol and/or amine.

“Thioether” means a monovalent moiety derived from an alkyl moiety by replacing one or more hydrogen atoms with an —SR group wherein R is alkyl.

Furthermore, the use of the term “consisting essentially of,” in referring to a method of treatment, means that the method substantially does not involve providing another therapy and/or another active agent in amounts and/or under conditions that would be sufficient to provide the treatment, and which are other than the therapies and/or active agents specifically recited in the claim. Similarly, the use of the term “consisting essentially of,” in referring to a kit for treatment, means that the kit substantially does not include another therapy and/or another active agent provided in amounts and/or under conditions that would be sufficient to provide the treatment, and which are other than the therapies and/or active agents specifically recited in the claim.

### DETAILED DESCRIPTION

In one embodiment, aspects of the present disclosure are directed to a process for preparing a pentaaza macrocyclic ring complex according to any of the Formulas herein. Specifically, aspects of the present disclosure are directed to a process for preparing a pentaaza macrocyclic ring complex according to any of Formulas (I)(a) or (I)(b) below (Formulas (I)(a) and (I)(b) are mirror image stereoisomers of one another, i.e. enantiomers):

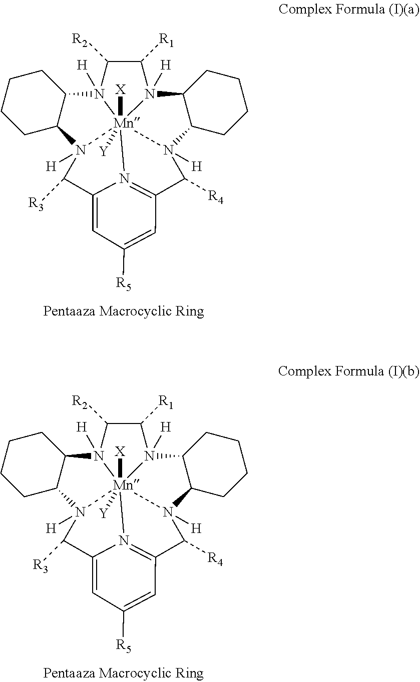

wherein X and Y are independently neutral or negatively charged ligands. For example, according to one embodiment, X and Y may independently be any of represent suitable ligands which are derived from any monodentate or polydentate coordinating ligand or ligand system or the corresponding anion thereof (for example benzoic acid or benzoate anion, phenol or phenoxide anion, alcohol or alkoxide anion).

According to one embodiment, X and Y may be independently selected from the group consisting of halo, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof, among other possibilities.

Furthermore, in one embodiment X and Y correspond to —O—C(O)—X₁, where each X₁ is independently substituted or unsubstituted phenyl or —C(—X₂)(—X₃)(—X₄);
each X₂ is independently substituted or unsubstituted phenyl, methyl, ethyl or propyl;
each X₃ is independently hydrogen, hydroxyl, methyl, ethyl, propyl, amino, —X₅C(═O)R₁₃ where X₅ is NH or O, and R₁₃ is C₁-C₁₈ alkyl, substituted or unsubstituted aryl or C₁-C₁₈ aralkyl, or —OR₁₄, where R₁₄ is C₁-C₁₈ alkyl, substituted or unsubstituted aryl or C₁-C₁₈ aralkyl, or together with X₄ is (═O); and
each X₄ is independently hydrogen or together with X₃ is (═O).

In one embodiment, X₁ is —C(—X₂)(—X₃)(—X₄) and each X₂, X₃, and X₄, in combination, corresponds to any of the combinations identified in the following table:

| Combination | X₂ | X₃ | X₄ |
|---|---|---|---|
| 1 | Ph | H | H |
| 2 | Ph | OH | H |
| 3 | Ph | NH₂ | H |

| | | |
|---|---|---|
| 4 | Ph | ═O |
| | | (X₃ and X₄ in |
| | | combination) |

| | | | |
|---|---|---|---|
| 5 | Ph | CH₃ | H |
| 6 | CH₃ | H | H |
| 7 | CH₃ | OH | H |
| 8 | CH₃ | NH₂ | H |

| | | |
|---|---|---|
| 9 | CH₃ | ═O |
| | | (X₃ and X₄ in |
| | | combination) |

Furthermore, in one embodiment, X₁ is C(—X₂)(—X₃)(—X₄), and X₃ is —X₅C(═O)R₁₃, such that the combinations of X₂, X₃ and X₄ include any of the combinations identified in the following table:

| Combination | X₂ | X₃ | X₄ |
|---|---|---|---|
| 1 | Ph | NHC(═O)R₁₃ | H |
| 2 | Ph | OC(═O)R₁₃ | H |
| 3 | CH₃ | NHC(═O)R₁₃ | H |
| 4 | CH₃ | OC(═O)R₁₃ | H |

where R₁₃ is C₁-C₁₈ alkyl, substituted or unsubstituted aryl or C₁-C₁₈ aralkyl, or —OR₁₄, where R₁₄ is C₁-C₁₈ alkyl, substituted or unsubstituted aryl or C₁-C₁₈ aralkyl.

In yet another embodiment, X and Y are independently selected from the group consisting of charge-neutralizing anions which are derived from any monodentate or polydentate coordinating ligand and a ligand system and the corresponding anion thereof.

In one embodiment, X and Yin any of the formulas herein are independently selected from the group consisting of fluoro, chloro, bromo and iodo anions. In yet another embodiment, X and Y in any of the formulas herein are independently selected from the group consisting of alkyl carboxylates, aryl carboxylates and arylalkyl carboxylates. In yet another embodiment, X and Y in any of the formulas herein are independently amino acids. According to one embodiment, X and Y may independently be chloro ligands. According to another embodiment, X and Y may independently be propionato ligands.

In one embodiment, in Formulas (I)(a) and (I)(b) above, R₁-R₄ are independently selected from group consisting of hydrogen and substituted or unsubstituted alkyl, such as any of methyl, ethyl, propyl, 2-propyl, butyl and pentyl.

In one embodiment, in Formulas (I)(a) and (I)(b) above, R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH₂)₂N(CH₂CH₃)₂, NH₂ and NO₂.

### Cyclization Stage

According to embodiments herein, the process comprising a cyclization stage, which generally comprises reacting a tetraamine product comprising a tetraamine compound of Formula (III)(a)(i) or Formula (III)(a)(ii) below, or salt thereof, with a diacetyl pyridine compound of Formula (III)(b) below, and a source of manganese (II) ion corresponding to the formula Mn(X)(Y). According to certain embodiments, the reaction is performed in the presence of a tertiary amine.

The tetraamine compounds of Formula (III)(a)(i) and (II)(a)(ii) are shown below (the tetraamine compounds of Formulas (Ill)(a)(i) and (II)(a)(ii) are mirror image stereoisomers, i.e. enantiomers, of one another):

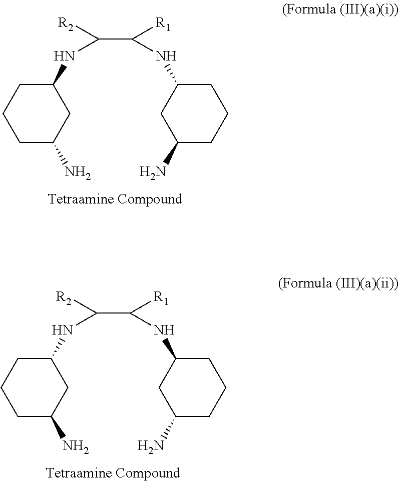

In Formulas (Ill)(a)(i) and (Ill)(a)(ii) above, R₁-R₂ are independently selected from group consisting of hydrogen and substituted or unsubstituted alkyl, as in Formulas (I)(a) and (I)(b) above. Furthermore, according to certain embodiments, the tetraamine compounds of Formula (III)(a)(i) and (Ill)(a)(ii) may be provided in the free base form as depicted above, or may be provided in the protonated salt form, such as the hydrochloride salt form (·4 HCl). Furthermore, according to certain embodiments, the tetraamine product comprises at least 99.0% optical purity of the tetraamine compound of Formula (III)(a)(i) or (Ill)(a)(ii). For example, according to certain embodiments, the tetraamine product comprising the tetraamine compound of Formula (III)(a)(i) or (Ill)(a)(ii) provided in the cyclization stage is optically pure, and contains less than 1%, less than 0.5%, and even less than 0.01% by weight of other stereoisomers, such as less than 1%, less than 0.5%, and even less than 0.01% by weight of its mirror image stereoisomer.

The diacetyl pyridine compound of Formula (III)(b) is shown below:

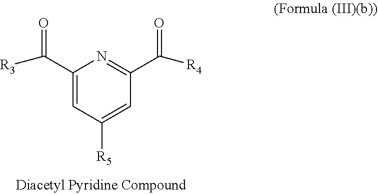

In Formula (III)(b) above, R₃ and R₄ are independently selected from group consisting of hydrogen and substituted or unsubstituted alkyl, as in Formulas (I)(a) and (I)(b) above. R₅ may be any specified for Formulas (I)(a) and (I)(b) above.

According to certain embodiments, the cyclization stage results in a bisimine compound of Formula (II)(a) or (II)(b) below (the bisimine compounds of Formulas (II)(a) and (II)(b) are mirror image stereoisomers, i.e. enantiomers, of one another).

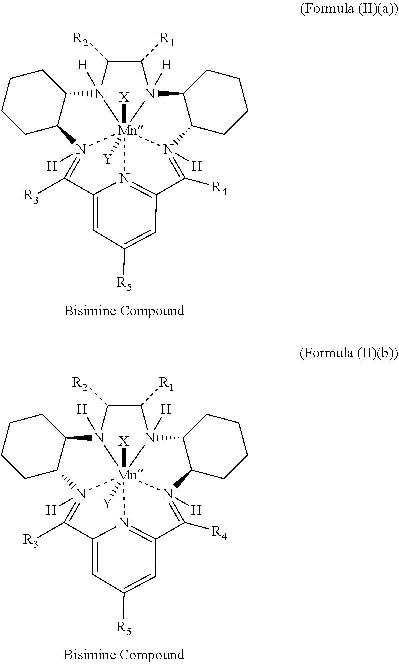

According to certain embodiments, cyclization stage product resulting from the cyclization stage comprises the bisimine compound of Formula (II)(a) or (II)(b) above having at least at least 95.0%, at least 97%, at least 98%, and/or even at least 99% optical purity. That is, in certain embodiments, the cyclization stage product resulting from the cyclization stage comprising the bisimine compound of Formula (II)(a) or (II)(b) is optically pure, and contains less than 1%, less than 0.5%, and even less than 0.01% by weight of other stereoisomers, such as less than 1%, less than 0.5%, and even less than 0.01% by weight of its mirror image stereoisomer.

In the bisimine compounds according to Formulas (II)(a) and (II)(b), X, Y, R₁-R₄, and R₅ may be any of those specified for Formulas (I)(a) and (I)(b) above. In another embodiment, X and Y in formulas (II)(a) and/or (II)(b) may be any listed as suitable for the pentaaza macrocyclic ring complex of Formulas (I)(a) and (I)(b) above. In one embodiment, the ligands X and Y present in the bisimine compounds of Formulas (II)(a) and/or (II)(b) may be the same or may be other than the X and/or Y present in the final pentaaza macrocyclic ring complex of Formula (I)(a) and/or Formula (II)(a). For example, an X and Y provided in the bisimine compound of Formula (II)(a) and/or (II)(b) may be altered during a step in the manufacturing process to become a different X and or Y in the resulting product. For example, according to one embodiment, X and Y provided in the bisimine compound of Formula (II)(a) and/or (II)(b) may independently be chloro, whereas X and Y provided in the pentaaza macrocyclic ring complex of Formula (I)(a) and/or Formula (I)(b) may independently be propionato. In another embodiment, X and Y in both the bisimine compound of Formula (II)(a) and/or (II)(b), and in the pentaaza macrocyclic ring complex of Formula (I)(a) and/or Formula (I)(b) are chloro.

According to one embodiment, the source of manganese (II) ion comprises, as counter-ions to the manganese ion, moieties corresponding to any of the ligands X and Y indicated as being suitable for the pentaaza macrocyclic ring complex according to Formulas (I)(a), (I)(b), (II)(a) and/or (II)(b) above. For example, in one embodiment, the source of manganese (II) ion comprises, as counter-ions to the manganese ion, moieties corresponding to any selected from the group consisting of halo, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins. For example, according to one embodiment, the source of manganese (II) ion can comprise any of Mn(II) chloride, Mn(II) bromide, and Mn(II) iodide. According to yet another embodiment, the source of manganese (II) ion can comprise any of Mn(II) propionate, Mn(II) acetate and Mn(II) nitrate.

According to one embodiment, the source of manganese (II) ions provided in the cyclization stage comprises one or more counter-ions corresponding to X and Y described for Formulas (I)(a), (I)(b), (II)(a) and/or (II)(b) above, and where the one or more counter ions include a counter-ion that is other than halo. For example, in one embodiment, the source of manganese (II) ion comprises, as a counter-ion to the manganese ion, a moiety corresponding to any selected from the group consisting of oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, and including any listed for Formulas (I)(a), (I)(b), (II)(a) and/or (II)(b) above that are not halo. For example, according to one embodiment, the source of manganese (II) ions provided in the cyclization stage comprises any of Mn(II) propionate, Mn(II) acetate and Mn(II) nitrate.

According to one embodiment, the bisimine compound of Formula (II)(a) and/or Formula (II)(b) comprises X and Y each independently corresponding to a first ligand moiety, and the process of preparing the pentaaza macrocyclic ring complex of Formula (I)(a) and/or (I)(b) further comprises the intermediate step of reacting the compound of Formula (II)(a) and/or (II)(b) with a source of a second ligand moiety, to provide a compound of Formula (II)(a) and/or (II)(b) having at least one of X and Y independently corresponding to the second ligand moiety. In one embodiment, the source of second ligands comprises a salt form of any of the moieties described for X and Y above, such as a sodium, calcium, magnesium or manganese salt of any such moieties. For example, according to one embodiment, the first ligand moiety comprises a chloro ligand (e.g. via reaction with Mn(II) chloride as a source of the first ligand moiety) and the second ligand moiety comprises a propionato ligand (e.g. via reaction sodium propionate as the second ligand moiety), such as to ultimately provide a compound according to Formula (I)(a) and/or Formula (I)(b) having X and Y independently as propionato. In one embodiment, the reaction with the source of the second ligand moiety may occur prior to initiating and/or performing the reduction stage, as described below. According to yet another embodiment, the manufacturing process can comprise reacting the compound of Formula (I)(a) or (I)(b) (e.g. post-reduction) with yet another source of ligand moiety to introduce yet another ligand moiety into the compound. For example, for a compound of Formula (I)(a) or (I)(b) having a first ligand moiety (e.g. X and Y as chloro), the compound may be reacted with a source of a second ligand moiety (e.g., a source of propionato ligand) to provide a compound of Formula (I)(a) or (I)(b) having the second ligand moiety (e.g., X and Y as propionato). Examples of processes for incorporating second ligand moiety (X and Y) into a compound of Formula (I)(a) or (I)(b) are provided in U.S. Pat. No. 9,738,670 to Keene et al., which is hereby incorporated by reference herein in its entirety (see, e.g., Example 9 on replacing chloro ligands with propionato ligands).

According to one embodiment, a process of preparing a bisimine complex of Formula (II)(a)(2) or (II)(b)(2) below is provided,

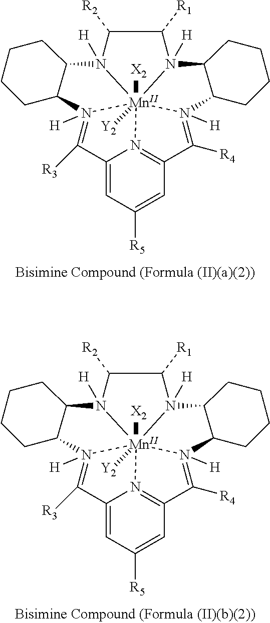

wherein X₂ and Y₂ are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,

According to certain embodiments, the process comprising reacting a Bisimine Compound of Formula (II)(a)(1) or (II)(b)(1) below having first ligands X₁ and Y₁, with a source of second ligands X₂ and Y₂ that are other than the first ligands, to provide the Bisimine Compound of Formula (II)(a)(2) or (II)(b)(2) where X₂ and Y₂ comprise the second ligands that are other than that of the first ligands,

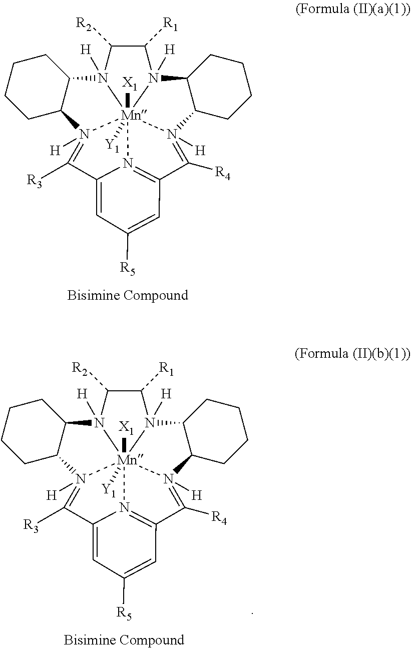

According to one embodiment, the first ligands X₁ and Y₁ comprise any X and Y disclosed herein, and the second ligands X₂ and Y₂ comprise any X and Y disclosed herein, and are other than X₁ and Y₁. In one embodiment, the second ligands X₂ and Y₂ are other than halo. For example, in one embodiment, the second ligands X₂ and Y₂ comprise any selected from the group consisting of alkyl carboxylates, aryl carboxylates and arylalkyl carboxylates, and the first ligands X₁ and Y₁ comprise any selected from the halo group, such as chloro. According to one embodiment, the source of second ligands X₂ and Y₂ comprises a source of propionato ligands. According to one embodiment, the source of the second ligands X₂ and Y₂ comprises a salt form of the ligands as described elsewhere herein such as sodium propionate. According to embodiments herein, the Bisimine Compound of Formula (II)(a)(1) or (II)(b)(1) (with the first ligands X₁ and Y₁) may be prepared according to any of the cyclization processes as described herein for preparing the Bisimine Compound of Formula (II)(a) or (II)(b) herein. According to further embodiments, the Bisimine Compound of Formula (II)(a)(2) or (II)(b)(2) (with the second ligands X₂ and Y₂) may be subject to any of the reduction processes as described herein, to prepare the pentaaza macrocyclic ring complex of Formula(I)(a) or (I)(b).

According to certain embodiments, the bisimine compound of any of Formulas (II)(a) and (II)(b) can be separately isolated from a reaction mixture used in the cyclization stage, such as for example to be used in subsequent synthetic steps to reach the pentaaza macrocyclic ring complexes of Formulas (I)(a) and (I)(b), or can be used for alternative synthetic routes. According to other embodiments, the bisimine compounds of any of Formulas (II)(a) and (II)(b) are not isolated from a reaction mixture used in the cyclization stage, but are maintained in the reaction mixture for further processing to achieve the pentaaza macrocyclic ring complexes of Formulas (I)(a) and (I)(b).

According to one embodiment, the cyclization stage is performed in the presence of a solvent comprising any one or more of 1-propanol, 2-propanol, ethanol, dimethyl formamide (DMF) and tetrahydrofuran (THF), and preferably 1-propanol. According to another embodiment, the tertiary amine provided in the cyclization stage comprises a tertiary amine base having a pKa above about 9, and even above about 10 and/or above about 11. According to one embodiment, the tertiary amine comprises the formula NR₁R₂R₃, wherein each of R₁, R₂ and R₃ comprise either substituted or unsubstituted cyclic or non-cyclic alkyl groups, with at least one of R₁, R₂ and R₃ being a non-cyclic alkyl group, and at least on one of R₁, R₂, and R₃ having 3 carbon atoms or less. According to certain embodiments, the tertiary amine base can comprise any of N,N-diisopropylamine (DIPEA), pempidine, and triethylamine. According to certain embodiments, the tertiary amine base has each of R₁, R₂, and R₃, as non-cyclic groups, such as with the tertiary amine base DIPEA. Additional bases that may be added to the cyclization stage can, in certain embodiments, comprising any of sodium hydroxide, lithium hydroxide, quinuclidine, triisobutylamine, 1,4-diazabicyclo[2.2.2]octane (DABCO) N-methylmorphonile (NMM), and morpholine. According to certain embodiments, a temperature of the reaction mixture during the cyclization stage is maintained in a range of from 50° C. to 130° C., such as in a range of from 60° C. to 120° C., and even about 97° C.

### Reduction Stage

According to certain embodiments, a reduction stage is performed to reduce the bisimine compound according to any of Formulas (II)(a) and (II)(b) to form the pentaaza macrocyclic ring complex according to any of Formulas (I)(a) and (I)(b). According to one embodiment, the reduction stage is performed after the diacetyl pyridine compound of Formula (III)(b) has reacted in the cyclization stage, such as for example after at least about 50% by weight, at least about 55% by weight, at least about 60% by weight, at least about 75% by weight, at least about 80% by weight, at least about 90% by weight, at least about 95% by weight, at least about 99% by weight, and even after substantially all of the diacetyl pyridine compound of Formula (III)(b) has reacted in the cyclization stage. According to yet another embodiment, the cyclization stage and reduction stage may be performed simultaneously with each other, such as for example after less than about 50% by weight, less than about 45% by weight, less than about 35% by weight, less than about 25% by weight, less than about 10% by weight, and even less than about 5% by weight of the diacetyl pyridine compound of Formula (III)(b) has reacted in the cyclization stage. According to one embodiment, the reduction stage is carried out to reduce the bisimine compound of Formula (II)(a) and/or Formula (II)(b) resulting from the cyclization stage, which bisimine compound comprises an optical purity of at least 95%, such as at least 97%, at least 98% and/or at least 99% optical purity.

According to one embodiment, the reduction stage is carried out by performing a catalytic hydrogenation reduction reaction on the bisimine compound of Formula (II)(a) and/or Formula (II)(b). According to certain embodiments, a catalytic hydrogenation reduction reaction has been found to advantageously provide the desired stereochemistry of the products, such that for example the bisimine compound of Formula (II)(a) is primarily and even entirely reduced to form the pentaaza macrocyclic ring complex of Formula (I)(a), and the bisimine compound of Formula (II)(b) is primarily and even entirely reduced to form the pentaaza macrocyclic ring complex of Formula (I)(b). For example, according to one embodiment, the reduction stage comprises performing a catalytic hydrogenation or other reduction reaction to provide a reduction stage product comprising the pentaaza macrocyclic ring complex of either Formula (I)(a) or Formula (I)(b) having at least 99% optical purity, such as at least 99.5%, at least 99.7% and/or even at least 99.8% optical purity. For example, in certain embodiments, the pentaaza macrocyclic ring complex of Formula (I)(a) or Formula (I)(b) resulting from the reduction stage is optically pure, and contains less than 1%, less than 0.5%, and even less than 0.01% by weight of other stereoisomers, such as less than 1%, less than 0.5%, and even less than 0.01% by weight of its mirror image stereoisomer. According to certain embodiments, the reduction stage product comprising the pentaaza macrocyclic ring complex of Formula (I)(a) or Formula (I)(b), and having the optical purity of at least 99%, is not subjected to any further optical purification processes after the reduction stage is performed. In other words, according to certain embodiments, the reduction stage provides the pentaaza macrocyclic ring complex product having the optical purity of at least 99%, without requiring further optical purification of the product.

According to one embodiment, the reduction stage comprises adding (i) catalytic hydrogenation reduction catalyst and (ii) a source of hydrogen (such as H₂ gas) to the reaction mixture comprising the bisimine compound of Formula (II)(a) and/or Formula (II)(b). For example, according to certain embodiments, the reduction stage, including addition of the catalyst and source of hydrogen, is performed at least 30 mins, at least 1 hour and/or at least 2 hours after the cyclization stage has begun, such as after the diacetyl pyridine compound of Formula (III)(b) has been added to react with the tetraamine compound of Formula (III)(a)(i) or (Ill)(a)(ii) in the cyclization stage. According to one embodiment, the reduction stage may be performed on the same reaction mixture as that used for the cyclization stage, or on the mixture resulting from the cyclization stage, such as for example by adding the catalytic hydrogenation catalyst and source of hydrogen to the same reaction mixture as the cyclization stage or resulting from the cyclization stage. According to another embodiment, the bisimine compound of Formula (II)(a) and/or (II)(b) may be isolated from the reaction mixture used to perform the cyclization stage, before the reduction stage is performed. According to yet another embodiment, a purification step may be performed to remove impurities from the bisimine compound of Formula (II)(a) and/or (II)(b) prior to performing the reduction reaction. For example, impurities such as sulfur-containing impurities, including dimethyl sulfide, may be removed. According to one embodiment, impurities may be removed from the bisimine compound of Formula (II)(a) and/or (II)(b) prior to performing the reduction stage to provide a bisimine product having less than 10 ppm, less than 5 ppm, less than 1 ppm, less than 0.5 ppm, and/or less than 0.1 pp, of sulfur-containing impurities.

According to one embodiment, the catalyst used in the catalytic reduction reaction comprises any suitable catalytic reduction catalyst such as any comprising palladium, platinum, iridium, ruthenium, rhodium or other catalytic reduction catalyst, such as any of palladium on carbon (wet or dry), palladium black, PtO₂, Raney Ni 2800, activated carbon, palladium on Al₂O₃, platinum on carbon, iridium on carbon, ruthenium on carbon, rhodium on carbon, sponge nickel catalyst, and palladium on BaSO₄. In one embodiment, the catalyst used in the catalytic reduction reaction comprises palladium on carbon. According to one embodiment, the catalytic reduction reaction is performed in the presence of a catalyst comprising from 3 wt % to 30 wt % palladium on carbon, such as from 5 wt % to 20 wt % palladium on carbon, such as 8 wt % to 15 wt % palladium on carbon. In one embodiment, the catalytic reduction reaction is performed in the presence of a catalyst comprising about 10 wt % palladium on carbon. In one embodiment, the catalytic reduction reaction is performed in the presence of from 1.25% w/w to 10% w/w (on an anhydrous basis) catalyst, such as from 2.5% w/w to 5% w/w (on an anhydrous basis) catalyst. In another embodiment, the catalytic reduction reaction is performed in the presence of about 2.5% w/w (on an anhydrous basis) catalyst. By “on an anhydrous basis” it is meant the loading of the catalyst as characterized on a dry or anhydrous basis, regardless of whether the catalyst is a “dry” or “wet” catalyst, as would be understood by a person of ordinary skill in the art. For example, for a “wet” catalyst (e.g. 50 wt % moisture), the loading amount of the catalyst to provide to the reduction reaction is determined as if the catalyst were anhydrous, and then the correct amount of “wet” catalyst can be provided by calculating the loading amount of wet catalyst that is the equivalent of the anhydrous catalyst, knowing the amount of moisture contained by the catalyst. According to certain embodiments, the catalytic reduction reaction is performed in the presence of catalyst comprising water. For example, in one embodiment the catalytic reduction reaction is performed in the presence of catalyst comprising from 1 wt % to 60 wt % of water, such as from 1.5% wt % to 55 wt % of water, from 20 wt % to 55 wt % of water, and even from 30 wt % to 55 wt % water. In one embodiment, the catalytic reduction reaction is performed in the presence of catalyst comprising at least 1 wt %, at least 1.5 wt %, at least 2.5 wt %, at least 3 wt %, at least 5 wt %, at least 10 wt %, at least 15 wt %, at least 20 wt %, at least 25 wt %, at least 30 wt %, at least 35 wt %, at least 40 wt %, at least 45 wt %, and/or at least 50 wt % of water.

According to certain embodiments, a temperature of the reaction mixture during the catalytic reduction stage is maintained in a range of from 50° C. to 130° C., such as in a range of from 50° C. to 120° C., including in a range of from 50° C. to 100° C., such as in a range of from 70° C. to 90° C., such as at a temperature of about 85° C. According to certain embodiments, the catalytic reduction reaction is performed in the presence of about 25 to 500 psi hydrogen, such as about 25 to 100 psi hydrogen, such as about 30 to 100 psi hydrogen. According to certain embodiments, the catalytic reduction reaction is performed in the presence of about 40 to 60 psi hydrogen, such as about 50 psi hydrogen. According to certain embodiments, the catalytic reduction stage is performed in the presence of a solvent, such as propanol, or other solvent described with respect to the cyclization stage above.

### Manganese-Containing Pentaaza Macrocyclic Ring Complex

In one embodiment, the pentaaza macrocyclic ring complex of Formula (I)(a) or (I)(b) is a compound represented by a formula selected from the group consisting of Formulae (V)-(XVI):

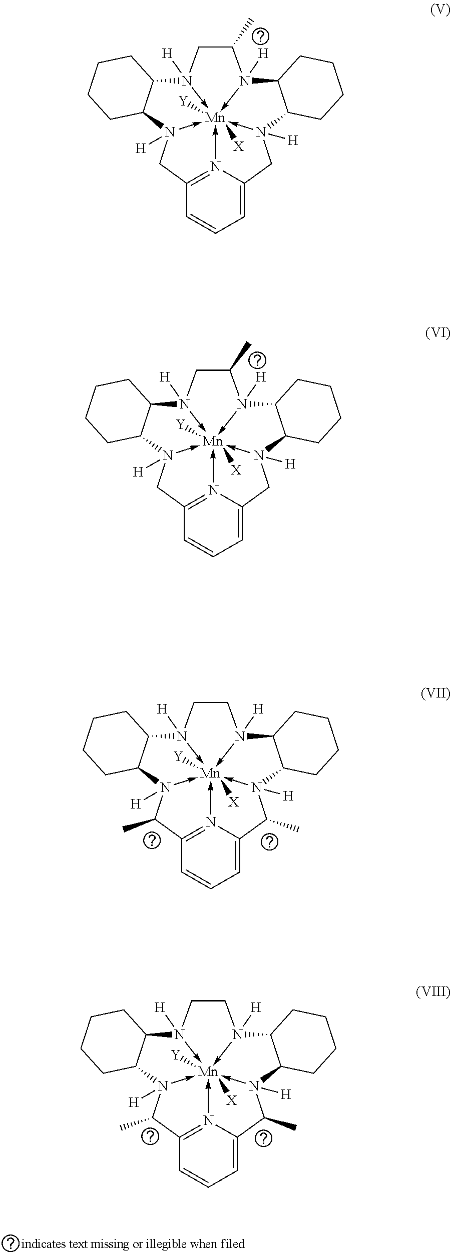

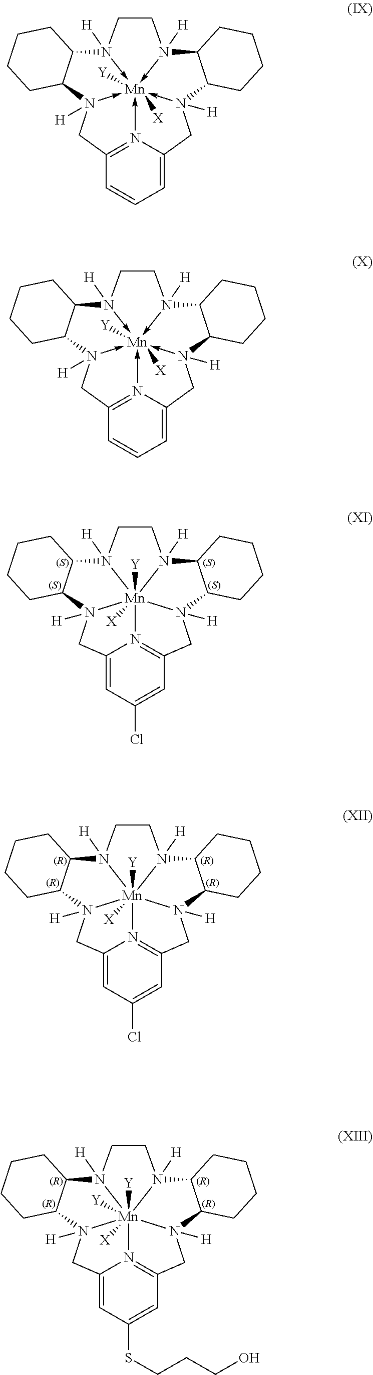

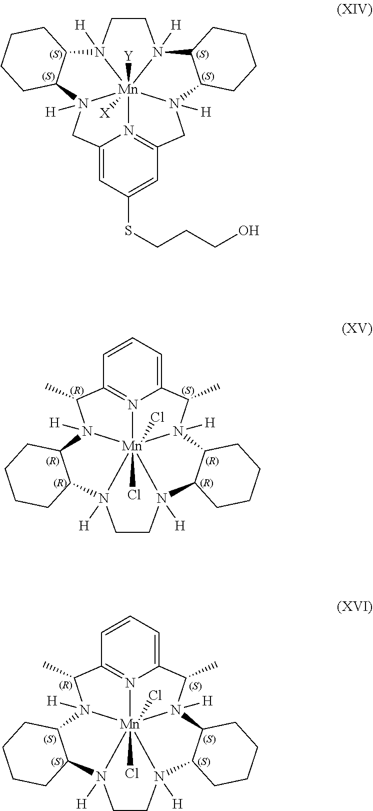

In one embodiment, X and Y in any of the formulae herein are as specified elsewhere herein, such as for example independently selected from the group consisting of fluoro, chloro, bromo and iodo anions. In yet another embodiment, X and Y in any of the formulae herein are independently selected from the group consisting of alkyl carboxylates, aryl carboxylates and arylalkyl carboxylates. In yet another embodiment, X and Y in any of the formulae herein are independently amino acids.

In one embodiment, the pentaaza macrocyclic ring complex corresponding to Formula (Ia) or (I)(b) is one of the complexes Formula (IE), such as (IE_{R1}), (IE_{S1}), (IE_{R2}), (IE_{S2}), (IE_{R3}), or (IE_{S3}):

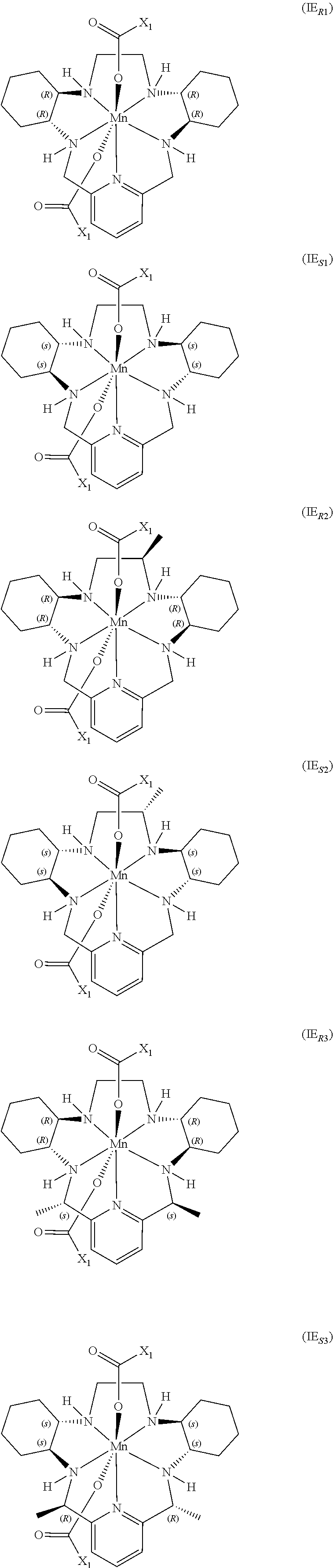

wherein
M is Mn^{+2 3}⁻;
each X₁ is independently substituted or unsubstituted phenyl or —C(—X₂)(—X₃)(—X₄);
each X₂ is independently substituted or unsubstituted phenyl or alkyl;
each X₃ is independently hydrogen, hydroxyl, alkyl, amino, —X₅C(═O)R₁₃ where X₅ is NH or O, and R₁₃ is C₁-C₁₈ alkyl, substituted or unsubstituted aryl or C₁-C₁₈ aralkyl, or —OR₁₄, where R₁₄ is C₁-C₁₈alkyl, substituted or unsubstituted aryl or C₁-C₁₈ aralkyl, or together with X₄ is (═O);
each X₄ is independently hydrogen or together with X₃ is (═O); and
the bonds between the transition metal M and the macrocyclic nitrogen atoms and the bonds between the transition metal M and the oxygen atoms of the axial ligands —OC(═O)X₁ are coordinate covalent bonds.

In one embodiment, within Formula (IE), and groups contained therein, in one group of compounds X₁ is —C(—X₂)(—X₃)(—X₄) and each X₂, X₃, and X₄, in combination, corresponds to any of the combinations identified in the following table:

| Combination | X₂ | X₃ | X₄ |
|---|---|---|---|
| 1 | Ph | H | H |
| 2 | Ph | OH | H |
| 3 | Ph | NH₂ | H |

| | | |
|---|---|---|
| 4 | Ph | ═O |
| | | (X₃ and X₄ in |
| | | combination) |

| | | | |
|---|---|---|---|
| 5 | Ph | CH₃ | H |
| 6 | CH₃ | H | H |
| 7 | CH₃ | OH | H |
| 8 | CH₃ | NH₂ | H |

| | | |
|---|---|---|
| 9 | CH₃ | ═O |
| | | (X₃ and X₄ in |
| | | combination) |

Furthermore, within embodiment (IE), and groups contained therein, in one group of compounds X₁ is C(—X₂)(—X₃)(—X₄), and X₃ is —X₅C(═O)R₁₃, such that the combinations of X₂, X₃ and X₄ include any of the combinations identified in the following table:

| Combination | X₂ | X₃ | X₄ |
|---|---|---|---|
| 1 | Ph | NHC(═O)R₁₃ | H |
| 2 | Ph | OC(═O)R₁₃ | H |
| 3 | CH₃ | NHC(═O)R₁₃ | H |
| 4 | CH₃ | OC(═O)R₁₃ | H |

where R₁₃ is C₁-C₁₈ alkyl, substituted or unsubstituted aryl or C₁-C₁₈ aralkyl, or —OR₁₄,
where R₁₄ is C₁-C₁₈ alkyl, substituted or unsubstituted aryl or C₁-C₁₈ aralkyl.

In one embodiment, the pentaaza macrocyclic ring complexes corresponding to Formula (I)(a) or (I)(b) can comprise any of the following structures:

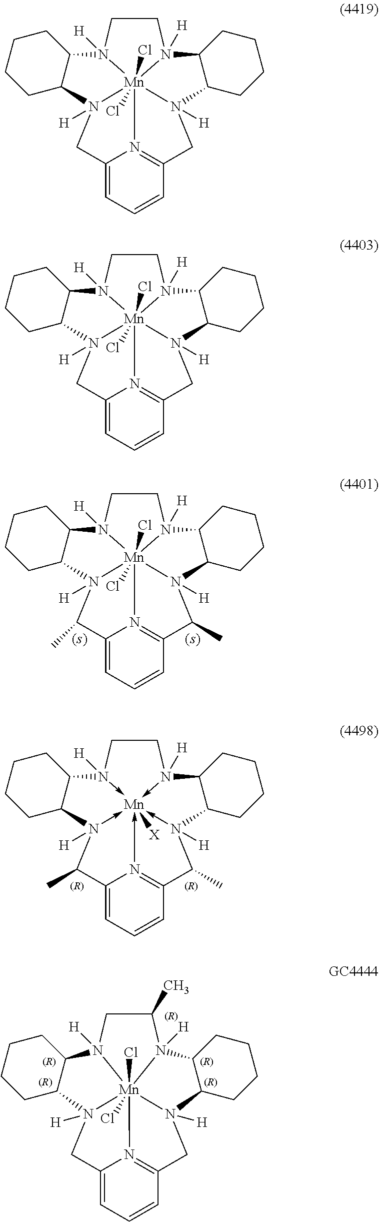

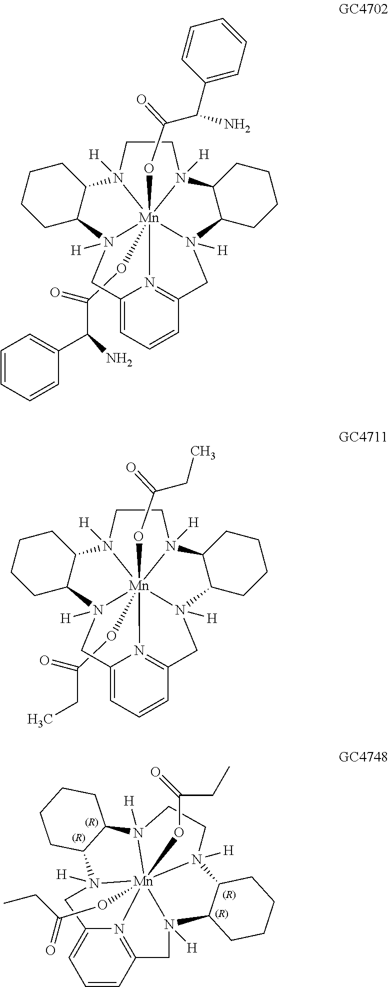

In another embodiment, the pentaaza macrocyclic ring complex corresponds to Formula (6) or Formula (7):

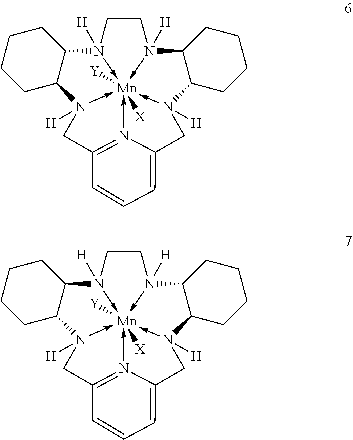

The chemical structures of 6 (such as the dichloro complex form described, for example, in Riley, D. P., Schall, O. F., 2007, Advances in Inorganic Chemistry, 59: 233-263) and of 7 herein (such as the dichloro complex form of 7), are identical except that they possess mirror image chirality; that is, the enantiomeric structures are non-superimposable. X and Y may correspond to any ligands described herein.

For example, the pentaaza macrocyclic ring complex may correspond to at least one of the complexes below:

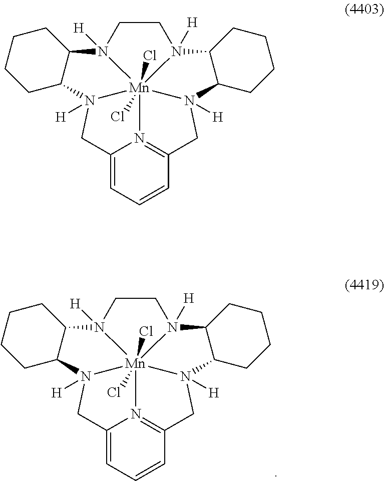

In yet another embodiment, the pentaaza macrocyclic ring complex may correspond to at least one of the complexes below, and/or an enantiomer thereof:

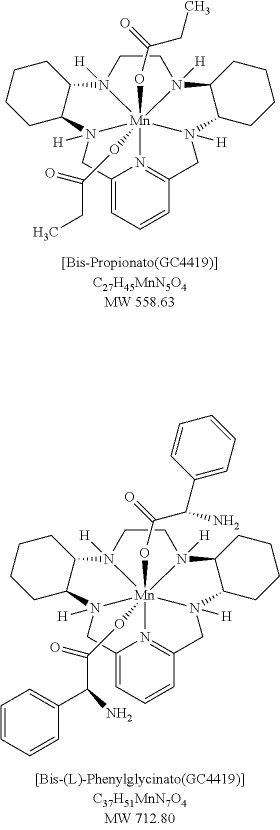

As used herein, the term “optical purity” refers to the amount of a compound having the depicted absolute stereochemistry, expressed as a percentage of the total amount of the depicted compound and its stereoisomers, including both enantiomers and diastereomers of the compound. In one embodiment, the optical purity of the pentaaza macrocyclic ring complex of Formula (I)(a) or (I)(b) is at least 99%, such as at least 99.5%, at least 99.7% and/or at least 99.8% optical purity. As used herein, the term “enantiomeric purity” refers to the amount of a compound having the depicted absolute stereochemistry, expressed as a percentage of the total amount of the depicted compound and its enantiomer. In one embodiment, the enantiomeric purity of the pentaaza macrocyclic ring complex is greater than 95%, more preferably greater than 98%, more preferably greater than 99%, and most preferably greater than 99.5%, such as at least 99.7% and even at least 99.8%. As used herein, the term “diastereomeric purity” refers to the amount of a compound having the depicted absolute stereochemistry, expressed as a percentage of the total amount of the depicted compound and its diastereomers. In one embodiment, the diastereomeric purity of the pentaaza macrocyclic ring complex is greater than 98%, more preferably greater than 99%, and most preferably greater than 99.5%, such as at least 99.7% and even at least 99.8%. Methods for determining optical purity, and including both diastereomeric and enantiomeric purity are well-known in the art. Diastereomeric purity can be determined by any analytical method capable of quantitatively distinguishing between a compound and its diastereomers, such as high-performance liquid chromatography (HPLC). Similarly, enantiomeric purity can be determined by any analytical method capable of quantitatively distinguishing between a compound and its enantiomer. Examples of suitable analytical methods for determining enantiomeric purity include, without limitation, optical rotation of plane-polarized light using a polarimeter, and HPLC using a chiral column packing material. According to one embodiment, the optical purity of the pentaaza macrocyclic ring complex is obtained via the processes described herein, without requiring any further optical purification methods, such as for example chiral chromatography, to be performed. For example, the cyclization and reduction stages can be performed to achieve the pentaaza macrocyclic ring complex having sufficient levels of optical purity, without performing any optical purification processes after the reduction stage, or between the cyclization and reduction stages. In one embodiment, the pentaaza macrocyclic ring complex resulting from the reduction stage may have sufficient levels of optical purity, such as the optical purities described herein, that subsequent optical purification steps are not required.

According to one embodiment, a dose of the pentaaza macrocyclic ring complex that is administered per kg body weight of the patient may be at least 0.1 mg/kg, such as at least 0.2 mg/kg. For example, the dose of the pentaaza macrocyclic ring complex that is administered per kg body weight of the patient may be at least 0.5 mg/kg. As another example, the dose of the pentaaza macrocyclic ring complex that is administered per kg body weight of the patient may be at least 1 mg/kg. In another example, the pentaaza macrocyclic compound that is administered per kg body weight may be at least 2 mg/kg, such as at least 3 mg/kg, and even at least about 15 mg/kg, such as at least 24 mg/kg and even at least 40 mg/kg. Generally, the dose of the pentaaza macrocyclic ring complex that is administered per kg body weight of the patient will not exceed 1000 mg/kg. For example the dose of the pentaaza macrocyclic ring complex that is administered per kg body weight of the patient may be in the range of from 0.1 to 1000 mg/kg, such as from 0.2 mg/kg to 40 mg/kg, such as 0.2 mg/kg to 24 mg/kg, and even 0.2 mg/kg to 10 mg/kg. As another example, the dose of the pentaaza macrocyclic ring complex that is administered per kg body weight may be in a range of from 1 mg/kg to 1000 mg/kg, such as from 3 mg/kg to 1000 mg/kg, and even from 5 mg/kg to 1000 mg/kg, such as 10 mg/kg to 1000 mg/kg. As another example, the dose of the pentaaza macrocyclic ring complex that is administered per kg body weight may be in a range of from 2 mg/kg to 15 mg/kg. As yet another example, the dose of the pentaaza macrocyclic ring complex that is administered per kg body weight may be in a range of from 3 mg/kg to 10 mg/kg. As another example, the dose of the pentaaza macrocyclic ring complex that is administered per kg body weight of the patient may be in the range of from 0.5 to 5 mg/kg. As yet a further example, the dose of the pentaaza macrocyclic ring complex that is administered per kg body weight of the patient may be in the range of from 1 to 5 mg/kg.

In one embodiment, the dose of the pentaaza macrocyclic ring complex may be at least 15 mg, at least 30 mg, at least 50 mg, at least 75 mg, at least 90 mg, at least 100 mg and/or at least 112 mg. The dose of pentaaza macrocyclic ring complex may also be administered over a predetermined period of infusion, such as a dosing rate for an infusion period of 15 minutes, 30 minutes, 45 minutes, 60 minutes, and/or a longer infusion duration. According to one embodiment, the pentaaza macrocyclic ring complex such as GC4419 may be administered at an infusion rate equivalent to at least 75 mg and/or at least 90 mg over course of an hour.

The dosing schedule of the pentaaza macrocyclic ring complex can similarly be selected according to the intended treatment. For example, in one embodiment, a suitable dosing schedule can comprise dosing a patient at least once per week, such as at least 2, 3, 4, 5, 6 or 7 days per week (e.g., daily), during a course of treatment. As another example, in one embodiment, the dosing may be at least once a day (qd), or even at least twice a day (bid).

### Methods of Treatment

Treatment of conditions including oral mucositis, cancer, or other conditions described herein includes achieving a therapeutic benefit, however the therapy may also be administered to achieve a prophylactic benefit. Therapeutic benefits generally refer to at least a partial eradication or amelioration of the underlying disorder being treated. For example, in a cancer patient, therapeutic benefit includes (partial or complete) eradication or amelioration of the underlying cancer. Also, a therapeutic benefit is achieved with at least partial, or complete, eradication or amelioration of one or more of the physiological symptoms associated with the underlying disorder such that an improvement is observed in the patient, notwithstanding the fact that the patient may still be afflicted with the underlying disorder. For prophylactic benefit, a method of the disclosure may be performed on, or a composition of the invention administered to, a patient at risk of developing cancer, or to a patient reporting one or more of the physiological symptoms of such conditions, even though a diagnosis of the condition may not have been made.

In general, any subject having, or suspected of having, a condition or disorder, may be treated using the compositions and methods of the present disclosure. Subjects receiving treatment according to the methods described herein are mammalian subjects, and typically human patients. Other mammals that may be treated according to the present disclosure include companion animals such as dogs and cats, farm animals such as cows, horses, and swine, as well as birds and more exotic animals (e.g., those found in zoos or nature preserves).

In accordance with one aspect of the present disclosure, methods are described herein for treating tissue damage resulting from a cancer treatment (e.g., radiation therapy or chemotherapy) delivered to a subject in need thereof. In accordance with another aspect of the present disclosure, methods are described herein for treating a human patient for tissue damage resulting from exposure to radiation. Thus, in various embodiments for example, the exposure to radiation in various embodiments may be an accidental radiation exposure, an unintentional radiation exposure, or an intentional radiation exposure. As noted above, treatment of tissue damage as described herein may include both inhibition (i.e., prophylaxis) and amelioration of any tissue damage that may result from an occurrence or activity. In general, the methods involve administering to the subject a therapeutically effective amount of the pentaaza macrocyclic ring complex. In one preferred embodiment, the complex is the dichloro complex form of Formula (GC4419), although other pentaaza macrocyclic ring complexes as described herein may also be used.

Treatment of tissue damage resulting from a cancer treatment or other radiation exposure in accordance with the methods described herein involves the administration of a therapeutically effective amount of the pentaaza macrocyclic ring complex, such as but not limited to, GC4419. In general, a range of therapeutically effective amounts may be used, depending, for example, on the compound selected and its safety and efficacy, the type, location, and severity of the tissue damage, among other factors. Examples of tissue damage that may be treated can include oral mucositis and other forms of tissue damage, including tissue damage affecting the mucosal lining of the upper and lower gastrointestinal tract.

According to yet another embodiment, the formulation can be used for treatment of cancers and/or tumors. Cancer and tumors generally refer to or describe the physiological condition in mammals that is typically characterized by unregulated cell growth. By means of the pharmaceutical formulations herein, various tumors can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and liver.

In one embodiment, the tumor or cancer is chosen from adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma, and teratoma. The tumor can be chosen from acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic, papillary serous adeno-carcinoma, pineal cell, pituitary tumors, plasmacytoma, pseudo-sarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

Thus, for example, the present disclosure provides methods for the treatment of a variety of cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, and teratocarcinoma.

For example, particular leukemias that can be treated with the formulations and methods described herein include, but are not limited to, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Lymphomas can also be treated with the formulations and methods described herein. Lymphomas are generally neoplastic transformations of cells that reside primarily in lymphoid tissue. Lymphomas are tumors of the immune system and generally are present as both T cell- and as B cell-associated disease. Among lymphomas, there are two major distinct groups: non-Hodgkin's lymphoma (NHL) and Hodgkin's disease. Bone marrow, lymph nodes, spleen and circulating cells, among others, may be involved. Treatment protocols include removal of bone marrow from the patient and purging it of tumor cells, often using antibodies directed against antigens present on the tumor cell type, followed by storage. The patient is then given a toxic dose of radiation or chemotherapy and the purged bone marrow is then re-infused in order to repopulate the patient's hematopoietic system.

Other hematological malignancies that can be treated with the combinations and methods described herein include myelodysplastic syndromes (MDS), myeloproliferative syndromes (MPS) and myelomas, such as solitary myeloma and multiple myeloma. Multiple myeloma (also called plasma cell myeloma) involves the skeletal system and is characterized by multiple tumorous masses of neoplastic plasma cells scattered throughout that system. It may also spread to lymph nodes and other sites such as the skin. Solitary myeloma involves solitary lesions that tend to occur in the same locations as multiple myeloma.

In one embodiment, the methods and formulations described herein are used to treat a cancer that is any of breast cancer, melanoma, oral squamous cell carcinoma, lung cancer including non-small cell lung cancer, renal cell carcinoma, colorectal cancer, prostate cancer, brain cancer, spindle cell carcinoma, urothelial cancer, bladder cancer, colorectal cancer, head and neck cancers such as squamous cell carcinoma, and pancreatic cancer. In yet another embodiment, the methods and formulations described herein are used to treat a cancer that is any of head and neck cancer and lung cancer.

As noted above, the diseases or conditions treated in accordance with the methods described herein may be any disease or condition that is/are treatable with the pentaaza macrocyclic ring complex. In one embodiment, for example, the disease or condition is selected from cancer, a cardiovascular disorder, a cerebrovascular disorder, a dermatological disorder, a fibrotic disorder, a gastrointestinal disorder, an immunological disorder, an inflammatory disorder, a metabolic disorder, a neurological disorder, an ophthalmic disorder, a pulmonary disorder, an infectious disease, and combinations thereof. By way of example, uses include the treatment of inflammatory and hyperproliferative skin diseases and cutaneous manifestations of immunologically-mediated illnesses, such as psoriasis, atopic dermatitis, contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophilias, lupus erythematosus, acne and alopecia greata; various eye diseases (autoimmune and otherwise) such as keratoconjunctivitis, vernal conjunctivitis, uveitis associated with Behcet's disease, keratitis, herpetic keratitis, conical cornea, dystrophia epithelialis corneae, corneal leukoma, and ocular pemphigus. In addition, reversible obstructive airway disease, which includes conditions such as asthma (for example, bronchial asthma, allergic asthma, intrinsic asthma, extrinsic asthma and dust asthma), particularly chronic or inveterate asthma (for example, late asthma and airway hyper-responsiveness), bronchitis, allergic rhinitis, and the like, can be treated, prevented, and/or ameliorated in accordance with the methods described herein. Other treatable diseases and conditions include inflammation of mucosa and blood vessels such as gastric ulcers, vascular damage caused by ischemic diseases and thrombosis. Moreover, hyperproliferative vascular diseases such as intimal smooth muscle cell hyperplasia, restenosis and vascular occlusion, particularly following biologically- or mechanically-mediated vascular injury, could be treated by the compounds described herein.

Still other treatable diseases and conditions include, but are not limited to, cardiac diseases such as post myocardial infarction, pulmonary diseases such as pulmonary muscle changes or remodeling and chronic obstructive pulmonary disease (COPD); ischemic bowel diseases, inflammatory bowel diseases, necrotizing enterocolitis, intestinal inflammations/allergies such as Coeliac diseases, proctitis, eosinophilic gastroenteritis, mastocytosis, Crohn's disease and ulcerative colitis; nervous diseases such as multiple myositis, Guillain-Barre syndrome, Meniere's disease, polyneuritis, multiple neuritis, mononeuritis and radiculopathy; septic shock and related refractory hypotension; endocrine diseases such as hyperthyroidism and Basedow's disease; arthritis (for example rheumatoid arthritis, arthritis chronica progrediente and arthritis deformans) and rheumatic diseases; hematic diseases such as pure red cell aplasia, aplastic anemia, hypoplastic anemia, idiopathic thrombocytopenic purpura, autoimmune hemolytic anemia, agranulocytosis, pernicious anemia, megaloblastic anemia and anerythroplasia; bone diseases such as osteoporosis; respiratory diseases such as sarcoidosis, fibroid lung and idiopathic interstitial pneumonia; skin disease such as dermatomyositis, leukoderma vulgaris, ichthyosis vulgaris, photoallergic sensitivity and cutaneous T cell lymphoma; circulatory diseases such as arteriosclerosis, atherosclerosis, aortitis syndrome, polyarteritis nodosa and myocardosis; collagen diseases such as scleroderma, Wegener's granuloma and Sjogren's syndrome; adiposis; eosinophilic fasciitis; periodontal disease such as lesions of gingiva, periodontium, alveolar bone and substantia ossea dentis; nephrotic syndrome such as glomerulonephritis; male pattern aleopecia or alopecia senilis by preventing epilation or providing hair germination and/or promoting hair generation and hair growth; muscular dystrophy; Pyoderma and Sezary's syndrome; Addison's disease; active oxygen-mediated diseases, as for example organ injury such as ischemia-reperfusion injury of organs (such as heart, liver, kidney and digestive tract) which occurs upon preservation, transplantation, organ failure (single or multi-), or ischemic disease (for example, thrombosis and cardiac infarction); dyskinetic disorders such as Parkinson's disease, neuroleptic-induced parkinsonism and tardive dyskinesias; intestinal diseases such as endotoxin-shock, pseudomembranous colitis and colitis caused by drug or radiation; renal diseases such as ischemic acute renal insufficiency and chronic renal insufficiency; pulmonary diseases such as toxinosis caused by lung-oxygen or drug (for example, paracort and bleomycins), lung cancer and pulmonary emphysema; ocular diseases such as cataracta, siderosis, retinitis, pigmentosa, senile macular degeneration, vitreal scarring and corneal alkali burn; dermatitis such as erythema multiforme, linear IgA ballous dermatitis and cement dermatitis; and others such as gingivitis, periodontitis, sepsis, pancreatitis, diseases caused by environmental pollution (for example, air pollution), aging, carcinogenesis, metastasis of carcinoma and hypobaropathy; diseases caused by histamine or leukotriene-C4 release; Behcet's disease such as intestinal-, vasculo- or neuro-Behcet's disease, and also Behcet's which affects the oral cavity, skin, eye, vulva, articulation, epididymis, lung, kidney and so on. Furthermore, the compounds of the invention are useful for the treatment and prevention of hepatic disease such as immunogenic diseases (for example, chronic autoimmune liver diseases such as autoimmune hepatitis, primary biliary cirrhosis and sclerosing cholangitis), partial liver resection, acute liver necrosis (e.g., necrosis caused by toxin, viral hepatitis, shock or anoxia), B-virus hepatitis, non-A/non-B hepatitis, cirrhosis (such as alcoholic cirrhosis) and hepatic failure such as fulminant hepatic failure, late-onset hepatic failure and “acute-on-chronic” liver failure (acute liver failure on chronic liver diseases), and for treatment of bacterial or viral infections such as influenza or HIV infection, and moreover are useful for various diseases because of their useful activity such as augmentation of chemotherapeutic effect, cytomegalovirus infection, particularly HCMV infection, anti-inflammatory activity, sclerosing and fibrotic diseases such as nephrosis, scleroderma, fibrosis (e.g., pulmonary fibrosis and lung fibrosis, including cryptogenic fibrosing alveolitis, idiopathic interstitial pneumonias, ideopathic pulmonary fibrosis, ideopathic mediastinal fibrosis, fibrosis complicating anti-neoplastic therapy, radiation therapy, and chronic infection, including tuberculosis and aspergillosis and other fungal infections), arteriosclerosis, congestive heart failure, ventricular hypertrophy, post-surgical adhesions and scarring, stroke, myocardial infarction and injury associated with ischemia and reperfusion, and the like.

### Pharmaceutical Formulations/Treatments

According to certain embodiments, formulations containing the pentaaza macrocyclic ring complex may be administered via a parenteral route (e.g., intravenous, intraarterial, subcutaneous, rectal, subcutaneous, intramuscular, intraorbital, intracapsular, intraspinal, intraperitoneal, or intrasternal). However, other routes of administration may also be possible therewith, such as oral, topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

Pharmaceutically acceptable additives and/or excipients for use in combination with the compositions of the present disclosure are well known to those of ordinary skill in the art and are selected based upon a number of factors: the particular compound(s) and agent(s) used, and its/their concentration, stability and intended bioavailability; the subject, its age, size and general condition; and the route of administration. Pharmaceutically acceptable additives for use in the pharmaceutical compositions described herein are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.) (Marcel Dekker, Inc., New York, New York, 1995), The Pharmacological Basis of Therapeutics, (Goodman & Gilman, McGraw Hill Publishing), Pharmaceutical Dosage Forms, (H. Lieberman et al., eds.) (Marcel Dekker, Inc., New York, New York, 1980), Remington's Pharmaceutical Sciences (A. Gennaro, ed., 19th ed.) (Mack Publishing, Easton, PA, 1995), The United States Pharmacopeia 24, The National Formulary 19, (National Publishing, Philadelphia, PA, 2000), and A. J. Spiegel et al., Use of Nonaqueous Solvents in Parenteral Products, Journal of Pharmaceutical Sciences, Vol. 52, No. 10, pp. 917-927 (1963).

Formulations for certain pentaaza macrocyclic ring complexes are also described in, for example, in U.S. Pat. Nos. 5,610,293, 5,637,578, 5,874,421, 5,976,498, 6,084,093, 6,180,620, 6,204,259, 6,214,817, 6,245,758, 6,395,725, and 6,525,041 (each of which is hereby incorporated herein by reference in its entirety).

The above-described pharmaceutical compositions including the pentaaza macrocyclic compound may additionally include one or more additional pharmaceutically active components. Suitable pharmaceutically active agents that may be included in the compositions according to aspects of the present invention include, for instance, antiemetics, anesthetics, antihypertensives, antianxiety agents, anticlotting agents, anticonvulsants, blood glucose-lowering agents, decongestants, antihistamines, antitussives, antineoplastics, beta blockers, anti-inflammatory agents, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism agents, anti-Alzheimer's Disease agents, antibiotics, anti-depressants, and antiviral agents. The individual components of such combinations may be administered either sequentially or simultaneously in separate or combined pharmaceutical formulations.

In yet another embodiment, a kit may be provided that includes the pentaaza macrocyclic ring complex, for treatment of a condition. For example, the kit may comprise a first vessel or container having therein a formulation comprising the pentaaza macrocyclic ring complex in an aqueous solution, such as an oral or injectable formulation of the pentaaza macrocyclic ring complex. The kit may further comprise a label or other instructions for administration of the active agents, recommended dosage amounts, durations and administration regimens, warnings, listing of possible drug-drug interactions, and other relevant instructions, such as a label instructing therapeutic regimens (e.g., dosing, frequency of dosing, etc.) corresponding to any of those described herein.

In yet another embodiment, the pentaaza macrocyclic ring complex can be administered as a part of a course of therapy that includes administration of a chemotherapeutic agent, such as for example a platinum-based chemotherapeutic agent (e.g., cisplatin). In chemotherapy, chemotherapeutic agents are administered to a patient to kill or control the growth of cancerous cells. A typical course of chemotherapy may include one or a plurality of doses of one or more chemotherapeutic agents, which can be administered over the course of days, weeks and even months. Chemotherapeutic agents can include at least one of: alkylating antineoplastic agents such as nitrogen mustards (e.g. cyclophosphamide, chlorambucil), nitrosoureas (e.g. n-nitroso-n-methylurea, carmustine, semustine), tetrazines (e.g. dacarbazine, mitozolimide), aziridines (e.g. thiotepa, mytomycin); anti-metabolites such as anti-folates (e.g. methotrexate and pemetrexed), fluoropyrimidines (e.g., fluorouracil, capecitabine), anthracyclines (e.g. doxorubicin, daunorubicin, epirubicin), deoxynucleoside analogs (e.g. cytarabine, gemcitabine, decitabine) and thiopurines (e.g., thioguanine, mercaptopurine); anti microtubule agents such as taxanes (e.g. paclitaxel, docetaxel); topoisomerase inhibitors (e.g. etoposide, doxorubicin, mitoxantrone, teniposide); antitumor antibiotics (e.g. bleomycin, mitomycin); and platins (e.g., cisplatin, carboplatin, oxaliplatin). For example, the chemotherapeutic agent may be selected from the group consisting of all-trans retinoic acid, arsenic trioxide, azacitidine, azathioprine, bleomycin, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, mechlorethamine, mercaptopurine, methotrexate, mitoxantrone, oxaliplatin, paclitaxel, pemetrexed, teniposide, tiguanine, valrubicin, vinblastine, vincristine, vindesine, and vinorelbine. The administration of many of the chemotherapeutic agents is described in the “Physicians' Desk Reference” (PDR), e.g., 1996 edition (Medical Economics Company, Montvale, N.J. 07645-1742, USA).

In yet another embodiment, the pentaaza macrocyclic ring complex can be administered as a part of a course of therapy that includes administration of a hormone therapy agent. Suitable hormone therapy agents may include, for example, any that target any one or more of the estrogen receptor pathway, progesterone receptor pathway, and the androgen receptor pathway, and can comprises any one or more of estrogen receptor inhibitors, estrogen receptor degraders/downregulators, selective estrogen receptor modulators (SERMs), aromatase inhibitors, GnRH agonists, androgen synthesis inhibitors, androgen receptor inhibitors, and selective progesterone receptor modulators (SPRMs). According to another embodiment, the hormone therapy agent comprises a SERM compound selected from the group consisting of tamoxifen, letrozole, clomifene, 4-hydroxytamoxifen, toremifene, raloxifene, nafoxidine, lasofoxifene, bazedoxifene, ospemifene, fulvestrant, brilanestrant, elacestrant, and derivatives, salts and/or prodrugs thereof. According to yet another embodiment, the hormone therapy agent comprises a SERM compound having a triphenylethylene structure, and/or a benzothiophene structure. According to yet a further embodiment, the hormone therapy agent comprises a SERM that is any one selected from the group consisting of tamoxifen, 4-hydroxytamoxifen, and derivatives, prodrugs and/or salts thereof. According to yet another embodiment, the hormone therapy agent targets the androgen receptor pathway, and comprises any one or more of an androgen receptor antagonist, an androgen synthesis inhibitor and an antigonadotropin. For example, the therapeutic agent that targets the androgen receptor pathway can comprise at least one selected from the group consisting of cyproterone acetate, megestrol acetate, chlormadinone acetate, spironolacone, oxendolone, osaterone acetate, flutamide, bicalutamide, nilutamide, topilutamide, enzalutamide, apalutamide, dienogest, drospirenone, medogestone, nomegestrol acetate, promegestone, trimegestone, ketoconazole, abiraterone acetate, seviteronel, aminoglutethimide, finasteride, dutasteride, episteride, alfatradial, cyproterone acetate, medrogestone, flutamide, nilutamide, bifluranol, leuprorelin, cetrorelix, allylestrenol, chlormadinone acetate, cyproterone acetate, gestonorone caproate, hydroxyprogesterone caproate, medroxyprogesterone acetate, megestrol acetate, osaterone acetate, oxendolone, estradiol, estradiol esters, ethinylestradiol, conjugated estrogens, diethylstilbestrol, and derivatives, salts and/or prodrugs thereof. According to yet another embodiment, the hormone therapy agent targets the progesterone receptor pathway, and comprises any one or more comprises a Type I, Type II or Type Ill selective modulator of progesterone (SPRM) that is at least one selected from the group consisting onapristone, mifepristone, lonaprisan, aglepristone, Org31710, Org31806, CDB-2914 and CDB-4124, and derivatives, salts and/or prodrugs thereof.

In yet another embodiment, the pentaaza macrocyclic ring complex can be administered as a part of a course of therapy that includes radiation therapy. In radiation therapy, a patient receives a dose or dose fraction of ionizing radiation to kill or control the growth of cancerous cells. The dose or dose fraction of radiation may be directed at a specific part of the body, and the beam of radiation may also be shaped according to a predetermined treatment regimen, to reduce deleterious effects on parts of the body not afflicted with cancer. A typical course of radiation therapy may include one or a plurality of doses or dose fractions of radiation, which can be administered over the course of days, weeks and even months. A total “dose” of radiation given during a course of radiation therapy typically refers to the amount of radiation a patient receives during the entire course of radiation therapy, which doses may be administered as dose “fractions” corresponding to multiple radiation exposures in the case where the total dose is administered over several sessions, with the sum of the fractions administered corresponding to the overall dose. In one embodiment, the radiation therapy can comprise any selected from the group consisting of gamma irradiation, proton therapy, heavy ion therapy, brachytherapy, radionuclide therapy, conformal radiation therapy, intensity modulated radiation therapy (IMRT), stereotactic body radiation therapy (SBRT), stereoablative radiation therapy, and gamma knife therapy, whether delivered as standard fractionation, hypofractionation, accelerated fractionation or decelerated fractionation and variations thereof.

A suitable overall dose of radiation to provide during a course of therapy can be determined according to the type of treatment to be provided, the physical characteristics of the patient and other factors, and the dose fractions that are to be provided can be similarly determined. In one embodiment, a dose fraction of radiation that is administered to a patient may be at least 1.8 Gy, such as at least 2 Gy, and even at least 3 Gy, such as at least 5 Gy, and even at least 6 Gy. In yet another embodiment, a dose fraction of radiation that is administered to a patient may be at least 10 Gy, such as at least 12 Gy, and even at least 15 Gy, such as at least 18 Gy, and even at least 20 Gy, such as at least 24 Gy. In general, a dose fraction of radiation administered to a patient will not exceed 54 Gy. Furthermore, it should be noted that, in one embodiment, a dose fraction delivered to a subject may refer to an amount delivered to a specific target region of a subject, such as a target region of a tumor, whereas other regions of the tumor or surrounding tissue may be exposed to more or less radiation than that specified by the nominal dose fraction amount. In yet another embodiment, the pentaaza macrocyclic ring complex is administered in combination with both a radiation therapy and a chemotherapy involving administration of a chemotherapeutic agent.

### EXAMPLES

The following non-limiting examples are provided to further illustrate aspects of the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Synthesis Examples

The following examples demonstrate synthesis of the pentaaza macrocyclic ring complex GC4403, which synthesis can be modified to provide for synthesis of other similar pentaaza macrocyclic ring complexes, such as those corresponding to Formula (I)(a) and (1)(b). In addition, other process parameters, steps, reactions and materials other than those specifically described in the Examples can be provided according to embodiments of the invention.

A schematic of an example of an overall process to synthesize a pentaaza macrocyclic ring complex according to Formula (I)(a) and/or (I)(b), and namely GC4403, is shown below. In a cyclization stage, tetraamine hydrochloride (M40400) is reacted with 2,6-Pyridinedicarboxaldehyde (2,6-PDCA) and MnCl₂, in the presence of DIPEA and 1-propanol, to generate the bisimine compound M40402 shown below. A catalytic reduction reaction with a palladium on carbon catalyst and H₂ is performed to reduce the bisimine compound to generate the compound GC4403 (referred to as M40403 in the schematic below). As would be understood by those of ordinary skill in the art, the mirror image compound (GC4419) can be generated by a same or similar reaction process, with a tetraamine starting material having the mirror image stereochemistry to that shown below, and by control of the stereochemistry of the compound during the reduction stage. Also, the compounds of Formula (I)(a) and/or (I)(b) having different R₁-R₄ groups can be provided by selecting tetraamine and/or diacetyl pyridine starting materials that are substituted with the desired groups (e.g. as shown in Formulas (Ill)(a)(i), (Ill)(a)(ii) and (Ill)(b)). Compounds of Formula (I)(a) and/or (I)(b) having different R₅ groups can be provided by selecting diacetyl pyridine materials that are substituted with the desired groups at the para position (e.g. as shown in Formula (III)(b)). Finally, as described herein, the final product (e.g. GC4403 below) can be reacted with a source of a second ligand moiety (e.g. sodium propionate) to provide axial ligands other than chloro, and such non-chloro ligands can also be introduced at different stages of the process, such as during the cyclization process (e.g. by providing a second ligand moiety as a counter-ion to Mn(II) in the place of chloro, such as MN(II)propionate), or after cyclization but before reduction of the bisimine (e.g. by reacting with a source of second ligand moiety such as sodium propionate).

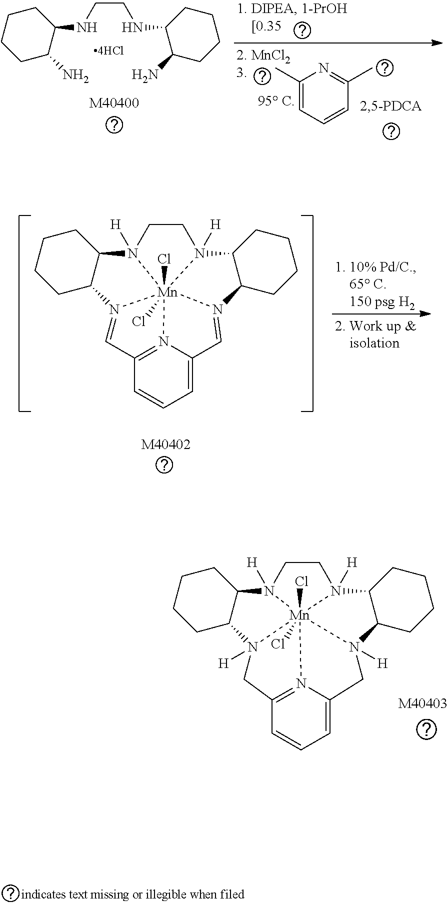

Chemicals, materials and methods: Ultra-dry manganese (II) chloride (99.99%, 42844) and palladium on carbon (10% Pd content, standard, reduced, nominally 50% H₂O content, 38304) were purchased from Alfa-/Esar. N,N′-Diisopropylethylamine (DIPEA, re-distilled, 99.5%, 38,764-9), 1-propanol (99.5+%, HPLC grade, 29,328-8) and filter agent, Celpure® P65 (USP-NF, pharmaceutical grade, 52,523-5) were purchased from Aldrich Chemical Co. 2,6-Pyridinedicarboxaldehyde (2,6-PDCA) was manufactured by ABC Laboratories, Columbia, Missouri. Tetraamine tetrahydrochloride M40400 was manufactured by either of CarboGen Laboratories AG, Aarau, Switzerland, Gateway Chemical Technology, St. Louis, Missouri or ABC Laboratories, Columbia, Missouri. 2-Propanol (99.9%, HPLC grade, A451-4), as well as all other solvents (HPLC-grade unless otherwise indicated) and reagents were purchased from VWR Scientific Products or Fisher Scientific and were of the finest grade available. “Reduced pressure” refers to operations carried out using a rotary evaporator and vacuum provided by a circulating water pump. “In vacuo” refers to high vacuum operations (50.5 torr), achieved with the use of an efficient, high capacity vacuum pump and an on-line dry-ice/2-propanol trap (capable of maintaining a temperature of ca. −40° C.). Elemental analysis was performed by Desert Analytics in Tucson, AZ (Mn and Na) or by Atlantic Microlab in Norcross, GA (all others). Reaction progress and product homogeneity was determined by HPLC analysis using a Varian ProStar system coupled to a Waters Symmetry-Shield™ RPS-18, 5 μm (4.6×250 mm) column. Solvent systems A and B consisted of 0.5 M LiCl/0.125 M TBAC in water and 1:4 (v/v) water:acetonitrile (CH₃CN), respectively. Elution was accomplished over a 20-min period using a 95:5 (v/v) A:B solvent mixture, run isocratically at a flow rate of 1.0 mL/min and UV detection at 265 nm. Samples for HPLC analysis were diluted/dissolved using mobile phase to afford typical analyte concentrations ca. 1 mg/mL and ca. 20 μL of this solution was injected.

### Cyclization Stage

An exemplary three-component template cyclization stage is described. Tetraamine hydrochloride M40400 (15.06 g, 37.6 mmol) was suspended in 1-propanol (110 mL, rendered a ca. 0.35 M mixture in M40400), thoroughly blanketed with Ar for 15 minutes, and stirred using a 19×35 mm Teflon blade (overhead stirrer, 700 rpm). DIPEA (26.2 mL, 4 equiv, 150.4 mmol) was added in three portions as a stream to the white suspension, which in less than a minute turned into a nearly colorless light syrup. After 10 min, MnCl₂ (4.78 g, 1 equiv, 38.0 mmol) was added in one portion. Thirty minutes after MnCl₂ addition, a nearly clear faint yellow solution had resulted (depending on the scale and efficiency of Ar purging the color may vary) and 2,6-pyridinedicarboxaldehyde (5.08 g, 1 equiv, 37.6 mmol) was added in one portion. The solution turned yellow-orange and heating commenced immediately, reaching 95° C. within 20 min. One hour later, 95% product conversion was detected by HPLC. Following a total of 4 h at 95±2° C., the brownish solution was cooled to near room temperature over 30 min. HPLC showed ca. 97% M40402 and the crude mixture was used as such immediately.

### Reduction Stage

An exemplary catalytic hydrogenation of bisimine M40402 is described. Once cool (ca. 30° C.), the mixture resulting from the cyclization stage described above was transferred to a Parr stainless steel reactor vessel for catalytic hydrogenation. The dark mixture was blanketed with Ar for 2-3 minutes, then 10% Pd/C (3.0 g total, 50% water wet, 10 wt % dry catalyst w/respect to M40400) was carefully added. The reactor was assembled and purged of air by pressurizing/depressurizing with N₂ (0 to 150 to 0 psig) five times. Next, the procedure was repeated three times using H₂ (0 to 150 to 0 psig), and finally the reactor was charged with H₂ (150 psig). The suspension was stirred (700 rpm) and heating commenced immediately. The set temperature, 85° C., was reached within 15 min. The reaction was stirred under pressure overnight for a total of 18 h. At this time, HPLC showed the reaction stream contained ca. 99% M40403. After bringing to room temperature over 1 h and purging with N₂ (5x as before), the reactor was disassembled and the suspension filtered through a bed of 1-propanol-washed celite (10 g) using a 25-50μ fritted funnel. The catalyst/celite bed was washed with 1-propanol (2×20 mL). The yellow solution was evaporated under reduced pressure until an opaque light yellow semi-solid remained (water bath temperature <35° C.). This material was stirred in water (700 mL, total solution volume ca. 800 mL) until dissolved (ca. 10 min). The pH of the faint yellow solution was measured as 5.82 and adjusted to 7.78 using 10% aq. NaOH (ca. 0.5 mL). NaCl (210 g, yields a ca. 25% solution in NaCl) was added to the slightly hazy light-yellow solution (total volume after NaCl addition ca. 900 mL). After stirring for 20 minutes, the off-white suspension was filtered using a 25-50μ fritted funnel. The cake was sucked dry under reduced pressure for approximately 5 minutes (at this time, no more foam dripped), transferred to a beaker, stirred (700 rpm using a 0.25×1 inch magnetic bar) in 20% aqueous NaCl (75 mL) for 15 min, and filtered as above. This washing/filtering procedure was repeated twice more in exactly the same manner. After the second wash, the left over off-white wet material was dried in vacuo (40° C., 17 h, 0.1 torr, cooled to room temperature over 1 h). Crude M40403 was isolated (19.06 g, 105% mass yield from M40400) as an off-white solid with a purity >99% by HPLC (This crude product contains ca. 5% NaCl and a water content of 5.8% corresponding to a 1.5 hydrated species). Once cool, the solid was broken down to a free-flowing powder prior to 2-propanol extraction. The solid was magnetically stirred (0.25×2 in bar, 800 rpm) in 200 mL of 2-propanol for 30 min and filtered through a 0.5-in bed of celite (pre-washed with 2-propanol immediately before filtration to avoid any possible water absorption). Water (100 mL) was added to the clear light yellow filtrate and the mixture was briefly swirled to homogenize. Solvents were then removed under reduced pressure (water bath temperature <35° C.) to afford an off-white solid that was further dried in vacuo (40° C., 18 h, 0.1 torr). M40403 was isolated as an off-white solid (16.63 g, 91% yield from M40400) with a purity >99% by HPLC. The material was broken down to a powder and stored in a freezer at 2-8° C.

Analytical determinations showed the following results HPLC Purity (TNAC): 99.55% M40403, 0.03% M40414 (a monoimine impurity), 0.10% M40500 (a double regioisomer impurity), 0.19% M40501 (a single regioisomer impurity).

### Parameter Studies

The following examples show the results for various tested parameters, including solvent, temperature, pressure, etc., for the above-described cyclization and reduction stages. Unless indicated otherwise, the parameters were evaluated using the processes described above for the cyclization and reduction stages.

INFLUENCE OF REACTION SOLVENT. Both steps in the above two-step process (cyclization+reduction) were tested using a variety of solvents. Class III solvents such as absolute ethanol (EtOH) and 1-propanol, and class II dimethylformamide (DMF) worked best. Other class Ill solvents tested include 2-propanol and tetrahydrofuran (THF), and class II methanol. All these proved less adequate either due to their boiling point (i.e., low boiling temperatures effectively delay reaction times while high boiling solvents become more difficult to remove during isolation) or poor solubility of starting materials (e.g. M40400, MnCl₂). Best results were obtained using 1-propanol due to its overall profile (i.e. relative low toxicity and reasonable boiling point [97° C.]), environmental and cost concerns, etc.

### General Reaction Scheme.

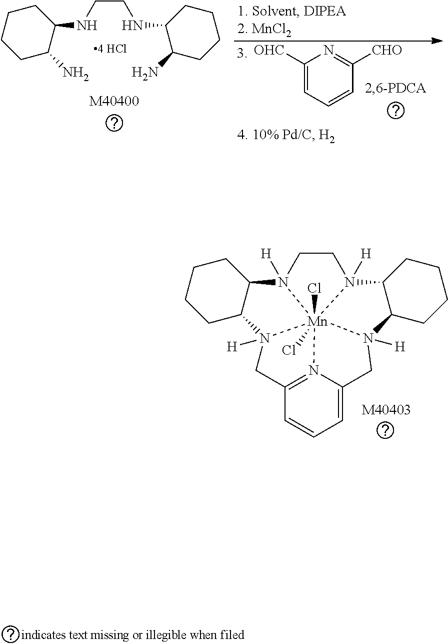

### Results Summary.

| | Parameter | | | | | | | | | | |
|---|---|---|---|---|---|---|---|---|---|---|---|
| | Cyclization Step, M40402 | | | | | Reduction Step, M40403 | | | | | |
| Reaction | Rxn | Bp | % | % | % | Rxn | % | % | % M40500 | % | % Mass |
| Solvent | Time | (° C.) | M40402 | M40527 | M40528 | Time | M40403 | M40414 | & M40501 | Other | Recovery |
| 1-PrOH | 4 | 97 | 96.6 | 0.1 | 0.1 | 18 | 99.8 | ND | 0.2 | ND | 91 |
| 2-PrOH | 4 | 82 | 96.9 | 0.1 | 0.1 | 18 | 99.6 | ND | 0.2 | 0.2 | 83 |
| EtOH | 4 | 78 | 96.5 | ND | 0.1 | 19 | 99.7 | ND | 0.2 | 0.1 | 92 |
| THF | 4 | 66 | 16 | NE | NE | Not Carried Out | | | | | |
| DMF | 4 | 153 | 95.9 | 1.2 | 1.0 | 4 | 95.6 | 0.3 | 0.7 | 3.4 | NE |

ND and NE stand for “not detected or <0.1%”, and “not established”, respectively.

The solvents were tested as described for the cyclization and reduction stages above, with the exception of tetrahydrofuran (THF) (99.9%, anhydrous). For the THE test, tetraamine hydrochloride M40400 (10.04 g, 25.1 mmol) was suspended in THE (70 mL, rendered a ca. 0.35 M in M40400 solution), and the mixture in the flask blanketed thoroughly with Ar for 10 minutes. DIPEA (17.5 mL, 4.0 equiv, 100.3 mmol) was added to the white suspension in two equal portions without noticeable change visually. After 10 min, and while stirring mechanically using a 1.5-inch Teflon™ paddle (500 rpm), MnCl₂ (3.17 g, 1 equiv, 25.2 mmol) was added in one portion. After 1 h of stirring, during which time the suspension never cleared, a considerable amount of MnCl₂ still remained in suspension. At this point, 2,6-pyridinedicarboxaldehyde (3.40 g, 1 equiv, 25.1 mmol) was added in one portion to the light pink mixture causing it to turn light orange. Heating commenced immediately and reached 65° C. within 15 min. Within this heating period, large brick-colored lumps formed suspended in an almost clear faint orange solution. After 4 h at 65 2° C., a sample of both solution and suspended solids was analyzed. HPLC showed ca. 16% M40402. The brownish solution was cooled to room temperature and the experiment was discontinued at this point. As referred to herein, M40402 is the bisimine compound having the proper stereochemistry to form M40403 (GC4403) (i.e. upon reduction of the bisimine double bonds), M40527 is a bisimine, single regioisomer impurity, M40528 is a bisimine, double regioisomer impurity, M40414 is a monoimine impurity, and M40500 and M40501 are double and single regioisomer impurities.

INFLUENCE OF BASE ON REACTIONS. Tests included organic and inorganic bases, such as diisopropylethyl-amine (DIPEA, pKa ˜11), triethylamine (TEA, pKa 10.75), 1,2,2,6,6-pentarnethylpiperidine (pempidine, pKa 11.25), 1-azabicyclo[2.2.2]octane (quinuclidine, pKa 10.95), 1,4-diazabicyclo[2.2.2]octane (triethylenediamine or Dabco™, pKa 2.97, 8.82), N-methylmorpholine (NMM, pKa 7.41), morpholine (pKa 8.36), and triisobutylamine (pKa˜11), as well as sodium and lithium hydroxides (pKa 15-16). The results seemed to indicate that the more nucleophilic (or less hindered) the character of the base, the greater the isomerization potential during template cyclization. Any isomerization that takes place during the template cyclization stage brings about a higher undesired isomer (e.g., M40500, M40501) content following catalytic hydrogenation. These scenarios may be best exemplified by comparing the use of DIPEA and lithium hydroxide as bases

### General Reaction Scheme.

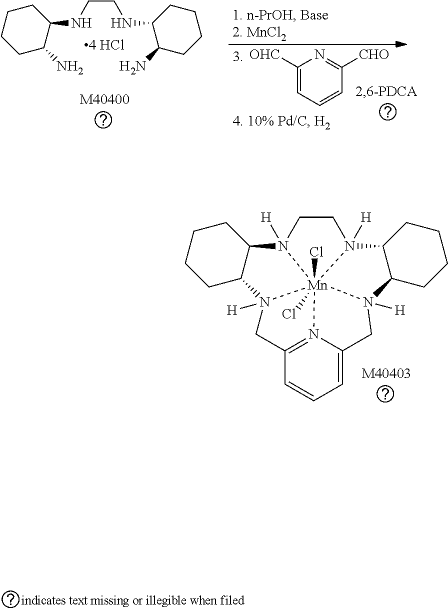

### Results Summary.

| | Parameter | | | | | | | | | |
|---|---|---|---|---|---|---|---|---|---|---|
| | Cyclization Step, M40402 | | | | Reduction Step, M40403 | | | | | |
| Base | Rxn | % | % | % | Rxn | % | % | % M40500 | % | % Mass |
| (4 equiv) | Time | M40402 | M40527 | M40528 | Time | M40403 | M40414 | M40501 | Other | Recovery |
| NaOH | 4 | 82 | 9 | 5 | 23 | 96.8 | 2.0 | 0.2 | 1.0 | 73 |
| LiOH | 5 | 63 | 26 | ND | Not Carried Out | | | | | |
| Triethylamine | 4 | 96 | 0.4 | 0.7 | 17 | 99.8 | ND | 0.2 | ND | 88 |
| DIPEA | 4 | 97 | 0.1 | 0.1 | 18 | 99.8 | ND | 0.2 | ND | 91 |
| Pempidine | 4 | 96 | 0.5 | 0.2 | 17 | 99.8 | ND | 0.2 | ND | 83 |
| Quinuclidine | 4 | 70 | 27 | 1 | 17 | 99.6 | ND | 0.3 | 0.1 | 88 |

| | | | | | |
|---|---|---|---|---|---|
| Triisobutylamine | 4 | 63 | ND | ND | Not Carried Out |
| DABCO | 4 | 84 | 14 | 1 | Not Carried Out |
| NMM | 4 | 51 | ND | ND | Not Carried Out |
| Morpholine | 4 | 13 | ND | ND | Not Carried Out |

ND stands for “not detected or <0.1%”.

The bases were tested as described for the cyclization and reduction stages above, with the exception of NaOH (98%, A.C.S. Grade) purchased from Aldrich. For the NaOH test, tetraamine hydrochloride M40400 (10.05 g, 25 mmol) was suspended in 1-propanol (70 mL, rendered a ca. 0.35 M in M40400), and the mixture in the flask blanketed thoroughly with Ar for 10 minutes. NaOH (98%, 4.08 g, 4.0 equiv, 100 mmol based on 98% purity) was added to the white suspension in one portion, which over time thinned somewhat. After 35 min, when the pellets had reacted, MnCl₂ (3.15 g, 1 equiv, 25 mmol) was added in one portion while stirring mechanically using a 1.5-inch Teflon paddle (400 rpm). The suspension thinned as the Mn(II) salt dissolved. After stirring for 20 min (600 rpm) most of the MnCl₂ had reacted and 2,6-pyridinedicarboxaldehyde (3.38 g, 1 equiv, 25 mmol) was added in one portion to the faint yellow suspension. Heating commenced immediately and reached 95° C. within 20 min. Within this heating period the suspension turned slightly thinner and deep orange in color. After 4 h at 95 2° C., and 13 h at room temperature (overnight), HPLC showed ca. 82% M40402. The mixture was used as such for hydrogenation.

A small amount of solids (NaCl) separated upon cooling the cyclization reaction. To prepare M40403, the liquid mixture was transferred to a 300-mL Parr vessel (with the aid of 20 mL of additional solvent, -0.28 M now) containing the “wet” palladium catalyst (2.0 g, 10 wt % with respect to M40400 on a dry basis). The reactor was assembled and purged of air by pressuri7ing/depressurizing with N₂ (0 to 150 to 0 psig) five times. Next, the procedure was repeated three times using H₂ (0 to 150 to 0 psig), and finally the reactor was charged with H₂ (150 psig). The suspension was stirred (700 rpm) and heating commenced immediately. The set temperature, 85° C., was reached within 15 min. The reaction was stirred under pressure overnight for a total of 23 h. At this time, HPLC showed the reaction stream contained ca. 95% M40403. After bringing to room temperature over 1 h and purging with N₂ (5x as before), the reactor was disassembled and the suspension filtered through a bed of 1-propanol-washed celite (10 g) using a 25-50μ fritted funnel. The catalyst/celite bed was washed with 1-propanol (2×20 mL). The light orange solution was evaporated under reduced pressure until a light yellow semi-solid remained (water bath temperature <35° C.). This material was stirred in water (460 mL, total solution volume ca. 500 mL) until dissolved (ca. 5 min). The pH of the yellow solution was measured as 7.89 and adjusted to 7.57 using 1% aq. HCl (ca. 0.2 mL). NaCl (160 g, yields a ca. 30% solution in NaCl) was added to the slightly hazy light yellow solution (total volume after NaCl addition ca. 580 mL). NaCl addition caused a yellow gum to separate which required vigorous stirring for 60 minutes to solidify into granules. This material was filtered using a 10-25μ fritted funnel. The cake was sucked dry under reduced pressure for approximately 10 min (at this time, no more foam dripped), transferred to a 150-mL beaker and stirred (700 rpm using a 0.25×1 inch magnetic bar) in 20% aqueous NaCl (50 mL) for 15 min, then filtered as above. This washing/filtering procedure was repeated twice more in exactly the same manner. After the third wash, the left over wet tan material was dried in vacuo (40° C., 17 h, 0.1 torr, cooled to room temperature over 1 h). Crude M40403 was isolated (10.52 g, 87% mass yield from M40400) as a tan solid. Once cool, the solid was broken down to a free-flowing powder prior to 2-propanol extraction. The solid was magnetically stirred (0.25×2 in bar, 800 rpm) in 130 mL of 2-propanol for 30 min and filtered through a 0.5-in bed of celite (pre-washed with 2-propanol immediately before filtration to avoid any possible water absorption). Water (65 mL) was added to the clear light yellow filtrate and the mixture was briefly swirled to homogenize. Solvents were then removed under reduced pressure (water bath temperature <35° C.) to afford an off-white solid that was then dried in vacuo (40° C., 19 h, 0.1 torr). M40403 was isolated as a tan solid (8.90 g, 73% yield from M40400) with a purity ca. 97% by HPLC.

INFLUENCE OF CATALYST ADDITION STAGE: DURING OR AFTER CYCLIZATION STEP. The effects of catalyst addition before (during cyclization) and after (after cyclization and prior to H₂ intake) step 3 were compared in the General Reaction Scheme below. Results obtained by incorporating the catalyst during the cyclization reaction seem to indicate a) an increase in the degree of isomerization and by-product formation and b) a decrease in the mass recovery of M40403 product.

### General Reaction Scheme.

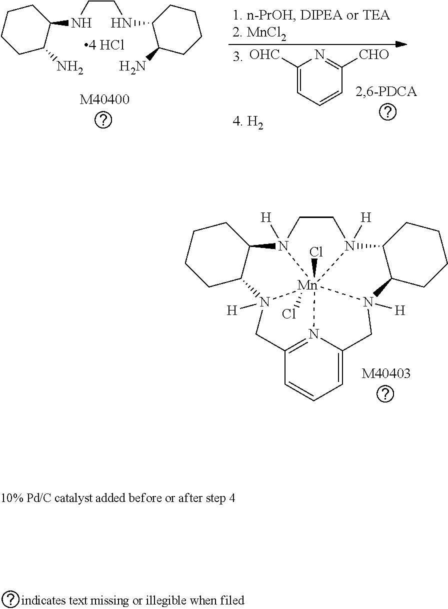

### Results Summary.

| | | Parameter | | | | | | | | | |
|---|---|---|---|---|---|---|---|---|---|---|---|
| | | Cyclization Step, M40402 | | | | Reduction Step, M40403 | | | | | |
| | Addition | Rxn | % | % | % | Rxn | % | % | % M40500 | % | Mass |
| Base | Stage | Time | M40402 | M40527 | M40528 | Time | M40403 | M40414 | M40501 | Other | Recovery |
| DIPEA | During | 4 | 96 | 0.4 | 0.2 | 18 | 97.9 | 1.3 | 0.1 | 0.7 | 78 |
| | After | 4 | 97 | 0.1 | 0.1 | 18 | 99.8 | ND | 0.2 | ND | 91 |
| TEA | During | 4 | 94 | 0.4 | 0.1 | 18 | 99.5 | 0.4 | 0.1 | ND | 76 |
| | After | 4 | 96 | 0.4 | 0.7 | 17 | 99.9 | ND | 0.1 | ND | 88 |

ND stands for “not detected or <0.1%”.

The addition after cyclization (prior to step 4 in the GENERAL REACTION SCHEME above), was as described for the cyclization and reduction stages described above.

The addition during cyclization (prior to step 3 in the General Reaction Scheme) was conducted as follows:

M40402: Tetraamine hydrochloride M40400 (10.05 g, 25.1 mmol) was suspended in 1-propanol (70 mL, rendered a ca. 0.35 M in M40400), and the mixture in the flask blanketed thoroughly with Ar for 10 minutes. Diisopropylethylamine (17.5 mL, 4.0 equiv, 100.5 mmol) was added to the white suspension in two portions, which over a few minutes turned into a nearly colorless light syrup. After 10 min, and while stirring mechanically using a 1.5-inch Teflon paddle (400 rpm), MnCl₂ (3.16 g, 1 equiv, 25.1 mmol) was added in one portion. Thirty minutes later, most MnCl₂ had reacted and a faint pink nearly clear solution resulted. At this point, the “wet” 10% Pd/C catalyst (2.0 g, 10% by wt of dry catalyst w/respect to M40400, ca. 50% water content) was added in one portion, followed by 2,6-pyridinedicarboxaldehyde (3.39 g, 1 equiv, 25.1 mmol) 2-3 minutes later. Heating commenced immediately and reached 95° C. within 20 min. After 4 h at 95±2° C., HPLC showed ca. 96% M40402. The mixture was cooled to room temperature over 0.5 h and used as such for hydrogenation.

M40403: The cyclization mixture was transferred to a 300-mL Parr vessel and the reactor assembled and purged of air by pressurizing/depressurizing with N₂ (0 to 150 to 0 psig) five times. Next, the procedure was repeated three times using H₂ (0 to 150 to 0 psig), and finally the reactor was charged with H₂ (150 psig). The suspension was stirred (700 rpm) and heating commenced immediately. The set temperature, 85° C., was reached within 15 min. The reaction was stirred under pressure overnight for a total of 18 h. At this time, HPLC showed the reaction stream contained ca. 96% M40403. After bringing to room temperature over 1 h and purging with N₂ (5× as before), the reactor was disassembled and the suspension filtered through a bed of 1-propanol-washed celite (10 g) using a 25-50μ fritted funnel. The catalyst/celite bed was washed with 1-propanol (2×20 mL). The yellow solution was evaporated under reduced pressure until an opaque light yellow semi-solid remained (water bath temperature <35° C.). This material was stirred in water (480 mL, total solution volume ca. 550 mL) until dissolved (ca. 20 min). The pH of the faint yellow solution was measured as 6.50 and adjusted to 7.62 using 10% aqueous NaOH (ca. 0.2 mL). NaCl (150 g, yields a ca. 25% solution in NaCl) was added to the slightly hazy light yellow solution (total volume after NaCl addition ca. 600 mL). After stirring for 20 minutes, the resulting light tan suspension was filtered using a 10-25μ fritted funnel. The cake was sucked dry under reduced pressure for approximately 5 minutes (at this time, no more foam dripped), transferred to a 150-mL beaker and stirred (700 rpm using a 0.25×1 inch magnetic bar) in 20% aqueous NaCl (50 mL) for 15 min, then filtered as above. This washing/filtering procedure was repeated twice more in exactly the same manner. After the third wash, the left over tan wet material (referred to as “crude M40403” in this experiment) was dried in vacuo (40° C., 24 h, 0.1 torr, cooled to room temperature over 1 h). Crude M40403 was isolated (12.3 g, 101% mass yield from M40400) as a light tan solid with a purity ca. 98% by HPLC. Once cool, the crusty cake was broken down to a free-flowing powder prior to 2-propanol extraction. The solid was magnetically stirred (0.25×1.5 in bar, 800 rpm) in 130 mL of 2-propanol for 30 min and filtered through a 0.5-in bed of celite (pre-washed with 2-propanol immediately before filtration to avoid any possible water absorption). Water (65 mL) was added to the clear light yellow filtrate and swirled to homogenize. Solvents were then removed under reduced pressure (water bath temperature 535° C.) to afford a tan solid that was then dried in vacuo (40° C., 18 h, 0.1 torr). M40403 was isolated as a light tan solid (9.45 g, 78% yield from M40400) with a purity ca. 98% by HPLC.

INFLUENCE OF TEMPERATURE DURING CYCLIZATION STAGE. The extent to which temperature influenced the cyclization step was explored. Lower temperatures decreased the rate of reaction thus allowing more time for side-reactions to occur. Higher temperatures (130° C., under pressure), although slightly faster in rate, also caused a slight increase in the extent of isomerization (relative to 97° C.) and a lower mass recovery.

### Results Summary.

| | Parameter | | | | | | | | | |
|---|---|---|---|---|---|---|---|---|---|---|
| Cyclization | Cyclization Step, M40402 | | | | Reduction Step, M40403 | | | | | |
| Temp. | Rxn | % | % | % | Rxn | % | % | % M40500 | % | Mass |
| (° C.) | Time | M40402 | M40527 | M40528 | Time | M40403 | M40414 | M40501 | Other | Recovery |
| 50 | 25 | 94.2 | 0.3 | 0.2 | 18 | 99.4 | 0.1 | 0.5 | ND | 90 |
| 97 | 4 | 96.6 | 0.1 | 0.1 | 18 | 99.8 | ND | 0.2 | ND | 91 |
| 130 | 2 | 95.6 | 1.1 | 0.5 | 17 | 99.1 | ND | 0.4 | 0.5 | 79 |

ND stand for “not detected or <0.1%”.

INFLUENCE OF REDUCTION AGENT: A) SODIUM BOROHYDRIDE, B) FORMATE-MEDIATED TRANSFER HYDROGENATION, C) CATALYTIC HYDROGENATION. The type of reduction agent/reduction process was also tested. The reaction profiles obtained showed considerable variation from method to method, mostly in terms of product purity and the extent of work up necessary for its isolation. Of the various methods tested catalytic hydrogenation proved to be the most efficient, convenient, and reliable. Borohydride reduction provided the least desirable outcome, and depending on the reaction temperature isomerization became a limiting factor in terms of product purity. Additionally, a considerable amount of quenching and work-up was necessary with borohydride reduction. Transfer hydrogenation was more reliable as far as HPLC purity and mass recovery of M40403 but also required considerable work-up, particularly to fully exchange formate for chloride in the final product. The sublimation of formate from the reaction was also inconvenient and hazardous. Upon condensation this material tended to obstruct glassware components such as condenser, etc.

### General Reaction Scheme.

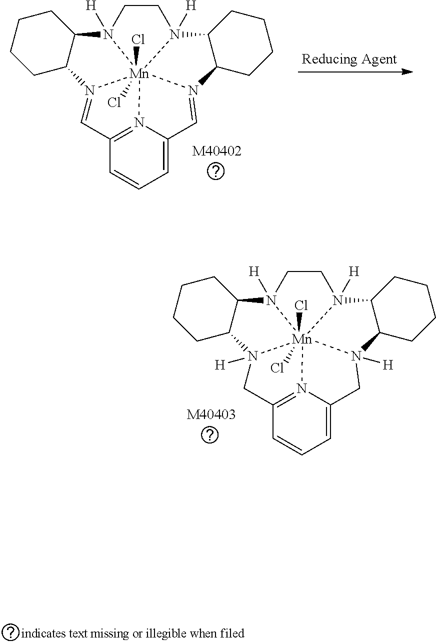

### Results Summary.

| | | Parameter | | | | | |
|---|---|---|---|---|---|---|---|
| | Reducing | Reduction Step, M40403 | | | | | |
| Reduction | Agent | Rxn Time | % | % | % M40500 | % | % Mass |
| Method | (equiv) | (hours) | M40403 | M40414 | M40501 | Other | Recovery |
| NaBH₄ | 4 | 4 | 98.5 | 0.7 | NE | NE | 93 |
| | | | | | | | (Ricerca) |
| Pd/C, Formate | 4 | 1 | 99.6 | ND | 0.3 | 0.1 | 84 |
| Pd/C, H₂ | Xs | 18 | 99.8 | ND | 0.2 | ND | 91 |

ND, NE and Xs stand for “not detected or <0.1%”, “not established” and “excess”, respectively. The HPLC method used at Ricerca was not suitable for separation of M40403 isomers and hence, the amounts of M40500 and M40501 were not initially established. Later on, it was learned that these had been indeed large.

For the catalytic hydrogenation approach, the cyclization and reduction stages as described above were performed.

For the sodium borohydride approach, the chemicals described above were used, adding in the NaBH₄ (99%, 21,346-2), which was purchased from Aldrich. Tetraamine hydrochloride M40400 (216 g, 0.539 mol, 0.32 M in M40400) was suspended in anhydrous DMF (4.7 L), and the flask purged thoroughly with Ar for 5 minutes. DIPEA (376 mL, 4 equiv., 2.156 mol) was added to the white suspension which first cleared, then quickly thickened to a glutinous mixture, and finally turned into a white suspension. After 15 min, and while stirring vigorously, MnCl₂ (anhydrous, 67.9 g, 0.539 mol) was added in one portion. Within minutes the pink solid dissolved, and a thinner faint orange mixture resulted (depending on the scale and efficiency of Ar purging the color may vary). 2,6-Pyridinedicarboxaldehyde (75 g, 0.539 mol) was added 45 min later causing a color change to deep orange. Heating commenced immediately, and the temperature was maintained at 95±5° C. for 5 h. The deep red-brown solution was cooled to near room temperature. The DMF solution was concentrated in vacuo (water bath temperature <40° C.) until a thick dark brown syrup resulted. Distilled water (10 L) was added and the mixture stirred for 30 min. NaCl (2.0 Kg) was then added to bring its concentration in the aqueous solution to 20%, and the mixture stirred for 1 h or until NaCl dissolves. After this period, the brick-colored suspension was filtered and the solids dried in vacuo (50° C., 0.3 torr, 16 h). The isolated material (274 g, 106% yield) is largely M40402, along with ca. 15% NaCl. The orange-brown bisimine has a purity of ca. 98% by HPLC and was used as such for the reduction step.

To prepare M40403, crude M40402 (1.02 g, 2.13 mmol) was partially dissolved in anhydrous EtOH (10 mL) under N₂ and cooled to ca. 0° C. using a salt-ice bath. NaBH₄ (0.32 g, 8.5 mmol, 2 equiv/double bond) was added in one portion causing some effervescence and a slight rise in temperature (up to 3-4° C.). The temperature was allowed to rise to that of the room 5 min after addition of NaBH₄. After 1 h at room temperature, MeOH (10 mL) was added dropwise from an addition funnel at a rate slow enough to maintain the reaction temperature between 25-30° C. (4-5 drops per second). The orange color suspension turned off-white within 15 min. At this point, LiCl (0.43 g, 10 mmol) was added with external cooling to prevent heating beyond 30° C. After 1 h, solvents were removed and the residue was dried in vacuo for 10 min. The resulting solids were taken up in water (25 mL), stirred until dissolved and NaCl (5 g) added to precipitate the crude product. After 15 min, the suspension was extracted with CH₂Cl₂ (4×15 mL). The pooled organic extracts were dried (MgSO₄), filtered and rendered dry under reduced pressure. The left over solids were re-dissolved in CH₂Cl₂ (1.5 mL) and added with a pipet onto a solution of MTBE-heptane (30 mL, 2:1 v/v). The precipitated material was filtered and dried in vacuo (40° C., 18 h, 0.5 torr) to afford 0.79 g (93% from M40402 containing 15% NaCl) of M40403 with a purity of 98.5% by HPLC (NaCl), 0.7% of which represents monoimine M40414.

For the transfer hydrogenation approach, the chemicals described above were used, adding in ammonium formate (Aldrich, 99%, 21,346-2) and 10% Pd/C (Johnson Matthey, Type A702032-10, 0.6% moisture). M40402 was prepared as described for the cyclization stage described above, but using anhydrous absolute ethanol (Aldrich, 45,983-6) as solvent. To prepare M40403, a 500-mL three-neck flask was charged the crude M40402 ethanol solution (containing 20.08 g of the bisimine, 41.9 mmol, and containing ca. 15% NaCl by wt.). The vermilion mixture was then blanketed with Ar for 10 minutes. Ar flow was interrupted momentarily as ammonium forniate (10.5 g, 166.5 mmol, ca. 2 equiv/bond) was carefully added in one portion, followed by 10% Pd/C (2.0 g, 10% by weight). Heating and magnetic stirring commenced immediately, and Ar flow ceased altogether. As the black suspension was heated (T=45-50° C.), a strong ammonia odor may be perceived coming out from the condenser outlet. The black suspension reached reflux within 20 min, and a coating of white salt started to deposit around the condenser's inner walls. After 0.5 hours at reflux, HPLC showed M40403 present in excess of 99.8%. Refluxed continued for an additional 0.5 hours and then the black suspension was allowed to cool to room temperature over 1 hour. The cool black suspension was cooled and filtered through a bed of EtOH-washed celite, and the solid bed washed with reagent EtOH (95%, 2×50 mL). The faint yellow solution was rendered dry under reduced pressure (water bath temperature <35° C. The left over solids were dissolved in water (200 mL) and the pH adjusted from 4.9 to 7.3 (pH meter) using 0.2 N aq. NaOH (ca. 3 mL, added in portions). NaCl (40 g) was added to the faint yellow aqueous solution and stirred for 15 min or until NaCl dissolved. The resulting milky white suspension was extracted with CH₂Cl₂ (3×150 mL) and the combined extracts dried for 15 min (MgSO₄, 20 g), filtered, and rendered dry under reduced pressure to afford light beige foam. This material was dissolved in 15-17 mL of CH₂Cl₂ and added pipet-wise onto a mixture of heptane:MTBE (1:2 v/v, 450 mL). The resulting white suspension was filtered immediately and the recovered solids dried in vacuo (0.5 tore, 40° C.) to constant weight (ca. 18 h). M40403 was isolated as a fine off-white powder (14.5 g, 84% from M40400) with a purity of 99.6% by HPLC.

### Influence of Catalyst Identity on Hydrogenation of M40402.

### General Reaction Scheme

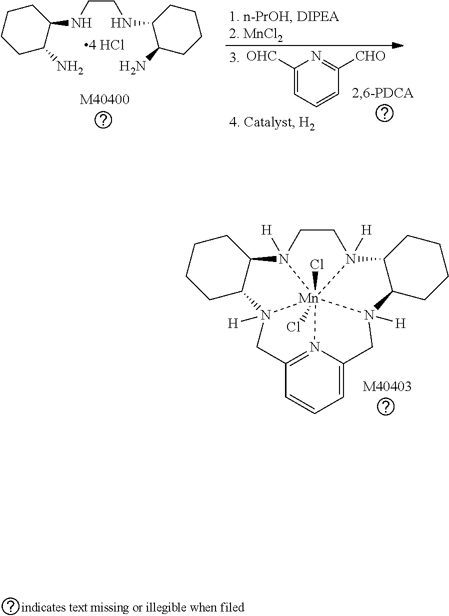

### Results Summary.

| | Parameter | | | | | |
|---|---|---|---|---|---|---|
| | Reduction Step, M40403 | | | | | |
| Hydro- | | | | % | | |
| genation | Rxn | % | % | M40500 | % | % Mass |
| Catalyst | Time | M40403 | M40414 | M40501 | Other | Recovery |
| 10% Pd/C | 18 | 99.8 | ND | 0.2 | ND | 91 |
| (wet) | | | | | | |
| 10% Pd/C | 17 | 99.6 | 0.1 | 0.2 | 0.2 | 93 |
| (dry) | | | | | | |
| Pd Black | 17 | 98.5 | 0.3 | 0.4 | 0.3 | NE |
| 10% Pt/C | 17 | 78.0 | 9.0 | 0.2 | 12.5 | NE |
| PtO₂ | 17 | 87.9 | 7.3 | 0.5 | 4.2 | NE |
| Raney Ni | 17 | 89.2 | 2.5 | 1.0 | 7.2 | NE |
| 2800 | | | | | | |
| 5% Rh/C | 23 | 99.3 | 0.3 | 0.4 | ND | NE |
| Activated | 17 | ND | ND | ND | 98.6 | NE |
| Carbon^{a} | | | | | (M40402) | |

ND and NE stand for “nox detected” and “not established”, respectively.
^{a}Essentially no bisimine reduction took place during hydrogenation stage.

To test the performance of a variety of catalysts, the hydrogenation process was performed as described for the reduction stage above but using various different catalysts. For 10% Pd/C (wet), 10% palladium on activated carbon catalyst (AMC-PMC, 2055C, 50% moisture) was used; for 10% Pd/C (dry), 10% palladium on activated carbon catalyst (AMC-PMC, 2055C, 1.5% moisture) was used; for Pd black, palladium black catalyst (Aldrich 20,583-4) was used; for 10% Pt/C, 10% platinum on carbon catalyst (Aldrich 20,598-8) was used; for PₜO₂, platinum (IV) oxide (Adam's catalyst; Aldrich 52,061-6) was used; for Raney Ni 2800, Raney Nickel catalyst (Aldrich 22,107-8) was used; for 5% Rh/C, 5% rhodium on carbon catalyst (Aldrich 20,616-4) was used; for activated carbon, activated carbon catalyst (Aldrich 24,227-6) was used.

### Influence of Metal Content of the Catalyst on Hydrogenation of M40402.

### General Reaction Scheme

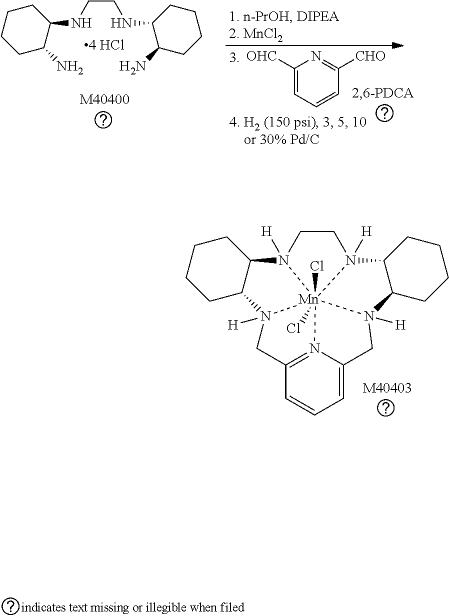

### Results Summary.

| | Parameter | | | | | |
|---|---|---|---|---|---|---|
| | Reduction Step, M40403 | | | | | |
| % | | | | % | | |
| Palladium | Rxn | % | % | M40500 | % | % Mass |
| on Carbon | Time | M40403 | M40414 | M40501 | Other | Recovery |
| 3 | 17 | 44.4 | 50.8 | 0.1 | ND | NE |
| 5 | 18 | 99.5 | 0.1 | 0.2 | 0.2 | NE |
| 10 | 17 | 99.6 | 0.1 | 0.2 | 0.2 | 93 |
| 30 | 17 | 96.7 | 1.0 | 0.4 | 1.9 | NE |

ND and NE stand for “not detected or <0.1%” and “not established”, respectively.

To test the impact of palladium loading on catalyst performance, the hydrogenation process was performed as described for the reduction stage above but using various palladium loadings of Pd/C (dry) catalyst replacing the 10% Pd/C (wet) catalyst. For 3% palladium on carbon, 3% Pd/C catalyst (AMC-PMC, 2055C, 1.7% moisture) was used; for 5% palladium on carbon, 5% Pd/C catalyst (AMC-PMC, 2055C, 1.6% moisture) was used; for 10% palladium on carbon, 10% Pd/C catalyst (AMC-PMC, 2055C, 1.5% moisture) was used; for 30% palladium on carbon, 30% Pd/C catalyst (Aldrich, 40,730-5, dry) was used.

### Palladium Catalyst Moisture Content (Dry Versus 50% Wet).

### Results Summary.

| | Parameter | | | | | |
|---|---|---|---|---|---|---|
| | Reduction Step, M40403 | | | | | |
| Moisture | | | | % | | |
| Content | Rxn | % | % | M40500 | % | % Mass |
| (wt %) | Time | M40403 | M40414 | M40501 | Other | Recovery |
| 1.5 | 17 | 99.6 | 0.1 | 0.2 | 0.2 | 93 |
| 50 | 18 | 99.8 | ND | 0.2 | ND | 91 |

The hydrogenation process was performed as described for the reduction stage above comparing 10% Pd/C (wet) and (dry) catalysts.

INFLUENCE OF TEMPERATURE ON HYDROGENATION OF M40402. An increase in temperature during the reduction stage generally increased the extent of hydrogenation of M40402 to M40403 over a given time period at a given H₂ pressure. At the highest temperature tested the formation of by-product isomers appeared to be modestly increased.

### Results Summary.

| | Parameter | | | | | |
|---|---|---|---|---|---|---|
| | Reduction Step, M40403 | | | | | |
| Hydrogenation | | | | % | | |
| Temperature | Rxn | % | % | M40500 | % | % Mass |
| (° C.) | Time | M40403 | M40414 | M40501 | Other | Recovery |
| 25 | 17 | 9.4 | 66.2 | ND | 24.4 | NE |
| 50 | 17 | 96.6 | ND | 0.8 | 2.6 | NE |
| 85 | 18 | 99.8 | ND | 0.2 | ND | 91 |
| 120 | 17 | 99.2 | ND | 0.6 | 0.2 | NE |

ND and NE stand for “not detected or <0.1% and “not established”, respectively.

The Experiments to test hydrogenation temperature were performed using the reduction stage described above, with the temperatures adjusted as per the table above.

INFLUENCE OF H₂ PRESSURE DURING HYDROGENATION. An increase in H₂ pressure during the reduction stage generally increased the extent of hydrogenation of M40402 to M40403 over a given time period at a given reaction temperature. At the highest pressure tested the formation of by-products appeared to be somewhat increased.

### General Reaction Scheme.

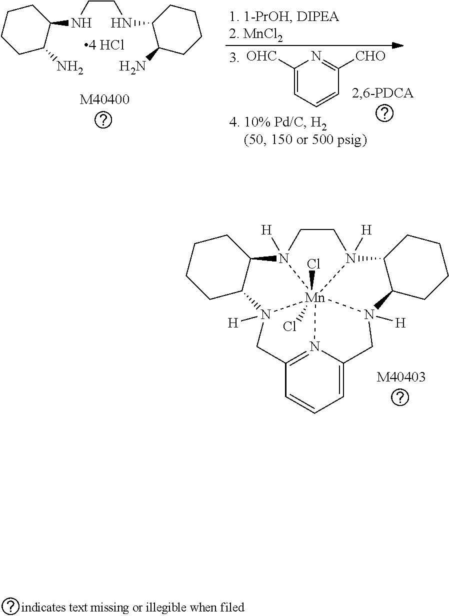

### Results Summary.

| | Parameter | | | | | |
|---|---|---|---|---|---|---|
| | Reduction Step, M40403 | | | | | |
| H₂ | | | | % | | |
| Pressure | Rxn | % | % | M40500 | % | % Mass |
| (psig) | Time | M40403 | M40414 | M40501 | Other | Recovery |
| 50 | 17 | 98.9 | ND | 0.6 | ND | ND |
| 150 | 18 | 99.8 | ND | 0.2 | ND | 91 |
| 500 | 17 | 97.8 | ND | 0.5 | 1.0 | ND |

ND stands for “not detected”.

The Experiments to test hydrogenation temperature were performed using the reduction stage described above, with the pressures set as per the table above.

### Embodiments

The Enumerated Embodiments 1-88 below set forth embodiments according to the disclosure.

Embodiment 1. A process of preparing a pentaaza macrocyclic ring complex of Formula (I)(a) or (I)(b) below:

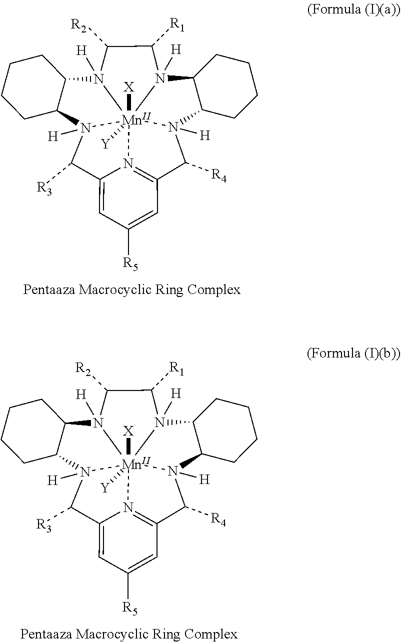

wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
the process comprising,
(A) in a cyclization stage, reacting a tetraamine product comprising a tetraamine compound of Formula (III)(a)(i) or Formula (III)(a)(ii) below, or a salt thereof, with a diacetyl pyridine compound of Formula (III)(b) below, and a source of manganese (II) ion corresponding to the formula Mn(X)(Y), in the presence of a tertiary amine base, wherein the tetraamine product comprises at least 99% optical purity of the compound of Formula (III)(a)(i) or Formula (III)(a)(ii) or salt thereof;

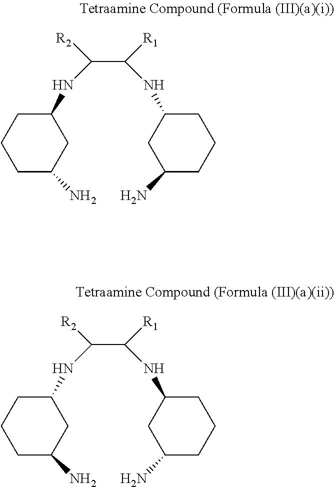

wherein R₁-R₄ are independently selected from group consisting of hydrogen and substituted or unsubstituted alkyl

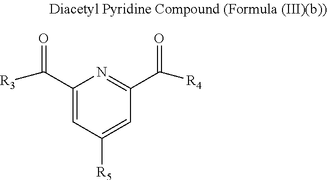

wherein R₃ and R₄ are independently selected from group consisting of hydrogen and substituted or unsubstituted alkyl, and
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
to provide a cyclization stage product comprising a bisimine compound of Formula (II)(a) or (II)(b) below having at least 95% optical purity

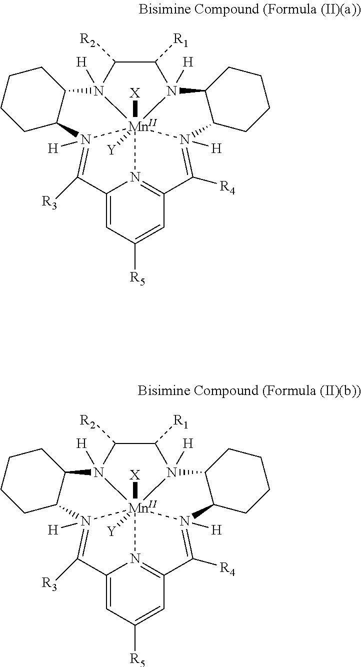

wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,and
(B) in a reduction stage performed after at least 50% by weight of the diacetyl pyridine compound of Formula (III)(b) has reacted in the cyclization stage, performing a catalytic hydrogenation reduction reaction on the bisimine compound of Formula (II)(a) or Formula (II)(b) to form a reduction stage product comprising the pentaaza macrocyclic ring complex of Formula (I)(a) or Formula (I)(b)having at least 99% optical purity.

Embodiment 2. The process according to Embodiment 1, wherein the bisimine product provided by the cyclization stage has the bisimine compound of Formula (II)(a) or (II)(b) having at least 97%, at least 98% and/or at least 99% optical purity.

Embodiment 3. The process according to any preceding Embodiment, wherein the reduction stage product has the pentaaza macrocyclic ring complex of Formula (I)(a) or Formula (I)(b) having least 99.5%, at least 99.7% and/or at least 99.8% optical purity.

Embodiment 4. The process according to any preceding Embodiment, wherein the reduction stage product comprising the pentaaza macrocyclic ring complex of Formula (I)(a) or Formula (I)(b) is not subjected to any further optical purification processes after the reduction stage is performed.

Embodiment 5. The process according to any preceding Embodiment, wherein the reduction stage is performed after at least 60% by weight, at least 75% by weight, at least 80% by weight, at least 90% by weight and/or at least 95% by weight of the diacetyl pyridine compound of Formula (III) has reacted in the cyclization stage.

Embodiment 6. The process according to any preceding Embodiment, wherein the reduction stage comprises adding a catalytic hydrogenation reduction catalyst and a source of hydrogen to the bisimine compound of Formula (II)(a) and/or (II)(b), and is performed at least 30 mins, at least 1 hour and/or at least 2 hours after the diacetyl pyridine compound is added in the cyclization stage.

Embodiment 7. The process according to any preceding Embodiment, further comprising isolating the bisimine compound of Formula (II)(a) or Formula (II)(b) prior to the reduction stage.

Embodiment 8. The process according to any preceding Embodiment, further comprising removing impurities from the bisimine product of the cyclization stage, prior to performing the reduction stage.

Embodiment 9. The process according to any preceding Embodiment, comprising removing sulfur-containing impurities.

Embodiment 10. The process according to any preceding Embodiment, comprising removing dimethyl sulfide impurities.

Embodiment 11. The process according to any preceding Embodiment, comprising removing impurities from the bisimine product to provide a product having less than 10 ppm, less than 5 ppm, less than 1 ppm, less than 0.5 ppm and/or less than 0.1 ppm of sulfur-containing impurities.

Embodiment 12. The process according to any preceding Embodiment, wherein X and Y are independently selected from the group consisting of halo, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof in any of Formulas (I)(a), (I)(b), (II)(a) and (II)(b).

Embodiment 13. The process according to any preceding Embodiment, wherein X and Y independently selected from the group consisting of fluoro, chloro, bromo and iodo anions in any of Formulas (I)(a), (I)(b), (II)(a) and (II)(b).

Embodiment 14. The process according to any preceding Embodiment, wherein X and Y independently selected from the group consisting of alkyl carboxylates, aryl carboxylates and arylalkyl carboxylates in any of Formulas (I)(a), (I)(b), (II)(a) and (II)(b).

Embodiment 15. The process according to any preceding Embodiment, wherein X and Y are chloro in any one of Formulas (I)(a), (I)(b), (II)(a) and (II)(b).

Embodiment 16. The process according to any preceding Embodiment, wherein X and Y are propionato in any one of Formulas (I)(a), (I)(b), (II)(a) and (II)(b).

Embodiment 17. The process according to any preceding Embodiment, wherein X and Y in any of Formulas (I)(a) and (I)(b) are other than X and Y in any of Formulas (II)(a) and (II)(b).

Embodiment 18. The process according to any preceding Embodiment, wherein X and Y in any of Formulas (I)(a) and (I)(b) are the same as X and Y in any of Formulas (II)(a) and (II)(b).

Embodiment 19. The process according to any preceding Embodiment, wherein X and Y in any of Formulas (II)(a) and (II)(b) are each chloro, and X and Y in any of Formulas (I)(a) and (I)(b) are each propionato.

Embodiment 20. The process according to any preceding Embodiment, wherein the source of manganese (II) ion comprises, as counter-ions to the manganese ion, moieties corresponding to the ligands X and Y in any one of Formulas (I)(a), (I)(b), (II)(a) and (II)(b).

Embodiment 21. The process according to any preceding Embodiment, wherein the source of manganese (II) ion comprises Mn (II) with counter-ions corresponding to any selected from the group consisting of halo, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

Embodiment 22. The process according to any preceding Embodiment, wherein the source of manganese (II) ion comprises any of Mn(II) chloride, Mn(II) bromide and Mn(II) iodide.

Embodiment 23. The process according to any preceding Embodiment, wherein the source of manganese (II) ion comprises any of Mn(II) propionate, Mn(II) acetate, and Mn(II) nitrate.

Embodiment 24. The process according to any preceding Embodiment, wherein the compound of Formula (II)(a) or (II)(b) comprises X and Y corresponding to a first ligand moiety, and wherein the process further comprises the intermediate step of reacting the compound of Formula (II)(a) or (II)(b) with a source of a second ligand moiety to provide a compound of Formula (II)(a) or (II)(b) having X and Y corresponding to the second ligand moiety, prior to performing the reduction stage (B).

Embodiment 25. The process according to any preceding Embodiment, wherein the first ligand moiety comprises a chloro ligand, and the second ligand moiety comprises a propionato ligand, and wherein the compound of Formula (I)(a) or (I)(b) comprises propionato ligands for X and Y.

Embodiment 26. The process according to any preceding Embodiment, wherein the process further comprises reacting the compound of Formula (I)(a) or (I)(b) with a source of another ligand moiety to introduce X and Y corresponding to the other ligand moiety.

Embodiment 27. The process according to any preceding Embodiment, wherein the process comprises reacting the pentaaza macrocyclic ring complex corresponding to the compound of formula (I)(a) or (I)(b) having X and Y ligands corresponding to chloro, with a source of a propionato ligand, to replace chloro with propionato as the X and Y ligands moiety in the compound of Formula (I)(a) or (I)(b).

Embodiment 28. The process according to any preceding Embodiment, wherein the pentaaza macrocyclic ring complex corresponding to the compound of formula (I)(a) or (I)(b) is any selected from the group consisting of Formulae (V)-(XVI) below:

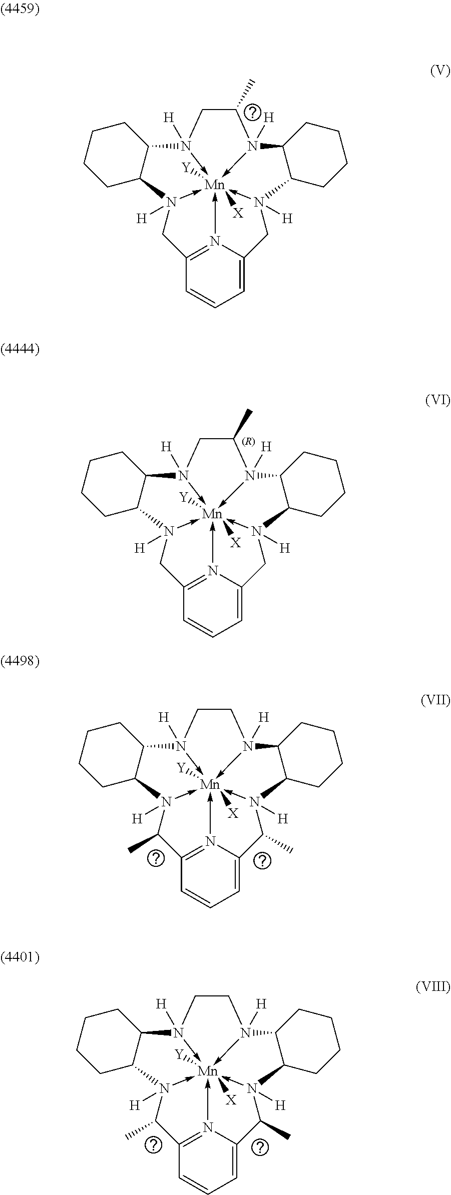

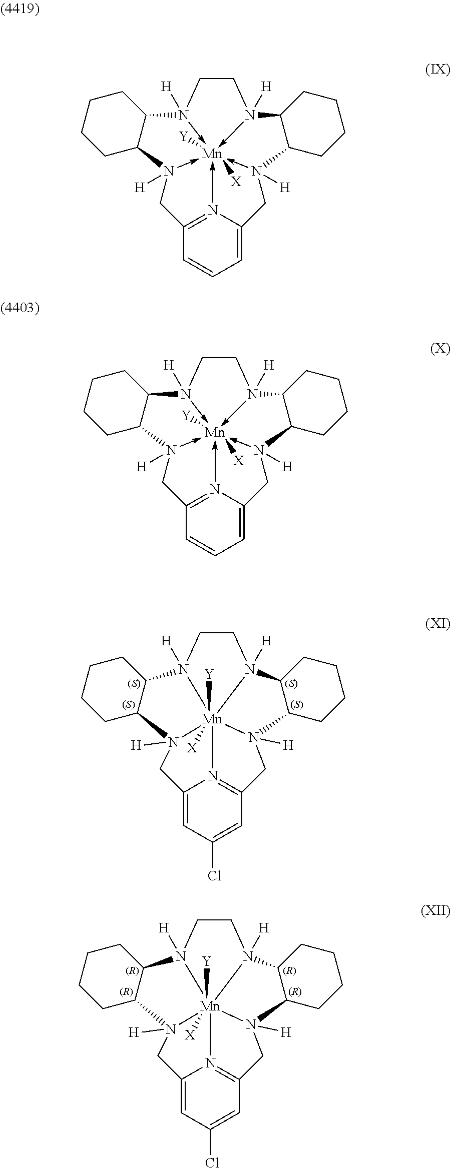

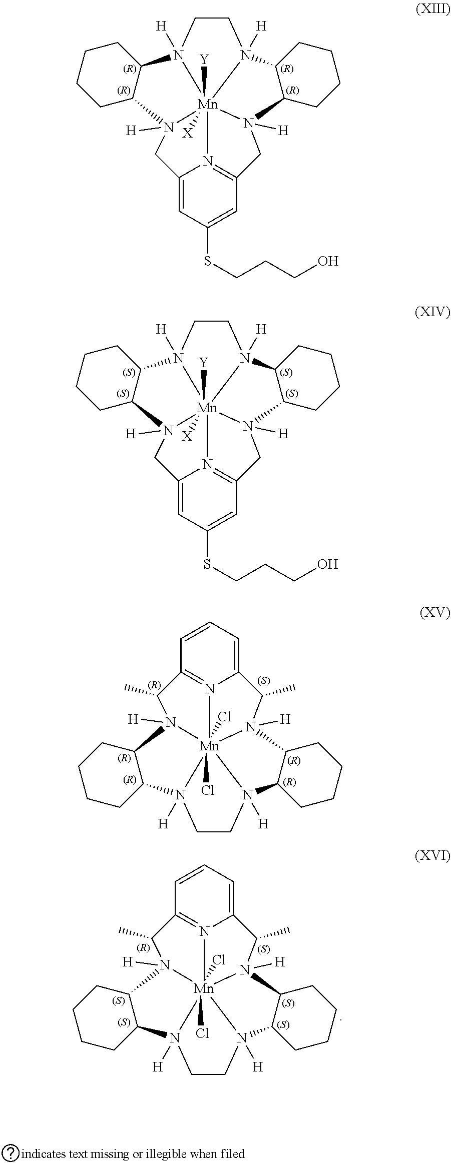

Embodiment 29. The process according to any preceding Embodiment, wherein the pentaaza macrocyclic ring complex corresponding to the compound of formula (I)(a) or (I)(b) is any selected from the group consisting of Formulae (IE_{R1}), (IE_{S1}), (IE_{R2}), (IE_{S2}), (IE_{R3}), and (IE_{S3}) below:

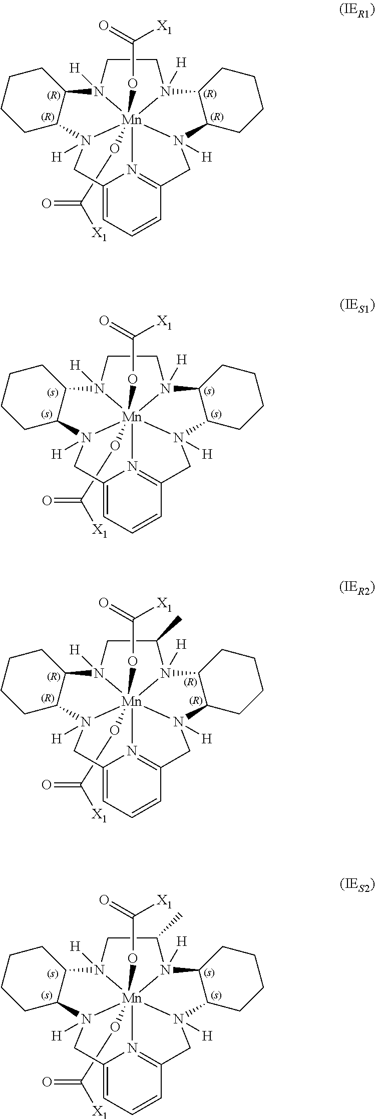

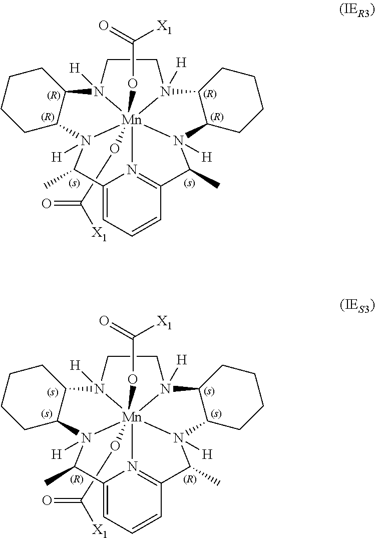

wherein
each X₁ is independently substituted or unsubstituted phenyl or —C(—X₂)(—X₃)(—X₄);
each X₂ is independently substituted or unsubstituted phenyl or alkyl;
each X₃ is independently hydrogen, hydroxyl, alkyl, amino, —X₅C(═O)R₁₃ where X₅ is NH or O, and R₁₃ is C₁-C₁₈ alkyl, substituted or unsubstituted aryl or C₁-C₁₈ aralkyl, or —OR₁₄, where R₁₄ is C₁-C₁₈alkyl, substituted or unsubstituted aryl or C₁-C₁₈ aralkyl, or together with X₄ is (═O);
each X₄ is independently hydrogen or together with X₃ is (═O); and
the bonds between the transition metal M and the macrocyclic nitrogen atoms and the bonds between the transition metal M and the oxygen atoms of the axial ligands —OC(═O)X₁ are coordinate covalent bonds.

Embodiment 30. The process according to any preceding Embodiment, wherein within any of the Formulae (IE_{R1}), (IE_{S1}), (IE_{R2}), (IE_{S2}), (IE_{R3}), and (IE_{S3}), X₁ is —C(—X₂)(—X₃)(—X₄) and each X₂, X₃, and X₄, in combination, corresponds to any of the combinations identified in the following table:

| Combination | X₂ | X₃ | X₄ |
|---|---|---|---|
| 1 | Ph | H | H |
| 2 | Ph | OH | H |
| 3 | Ph | NH₂ | H |

| | | |
|---|---|---|
| 4 | Ph | ═O |
| | | (X₃ and X₄ in |
| | | combination) |

| | | | |
|---|---|---|---|
| 5 | Ph | CH₃ | H |
| 6 | CH₃ | H | H |
| 7 | CH₃ | OH | H |
| 8 | CH₃ | NH₂ | H |

| | | |
|---|---|---|
| 9 | CH₃ | ═O |
| | | (X₃ and X₄ in |
| | | combination) |

Embodiment 31. The process according to any preceding Embodiment, wherein X₁ is C(—X₂)(—X₃)(—X₄), and X₃ is —X₅C(═O)R₁₃, such that the combinations of X₂, X₃ and X₄ include any of the combinations identified in the following table:

| Combination | X₂ | X₃ | X₄ |
|---|---|---|---|
| 1 | Ph | NHC(═O)R₁₃ | H |
| 2 | Ph | OC(═O)R₁₃ | H |
| 3 | CH₃ | NHC(═O)R₁₃ | H |
| 4 | CH₃ | OC(═O)R₁₃ | H |

where R₁₃ is C₁-C₁₈ alkyl, substituted or unsubstituted aryl or C₁-C₁₈ aralkyl, or —OR₁₄,
where R₁₄ is C₁-C₁₈ alkyl, substituted or unsubstituted aryl or C₁-C₁₈ aralkyl.

Embodiment 32. The process according to any preceding Embodiment, wherein the pentaaza macrocyclic ring complex corresponding to the compound of formula (I)(a) or (I)(b) is any selected from the group below:

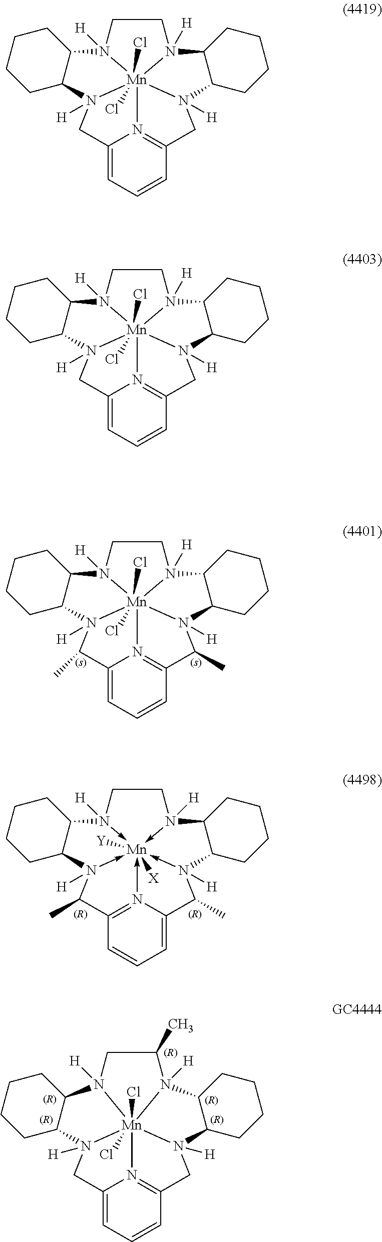

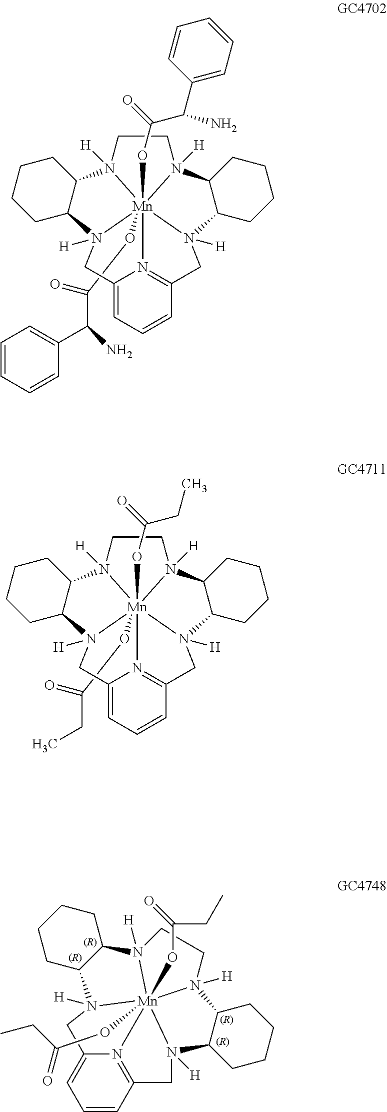

Embodiment 33. The process according to any preceding Embodiment, wherein the pentaaza macrocyclic ring complex corresponding to the compound of formula (I)(a) or (I)(b) is any selected from the group of Formula (6) or Formula (7) below:

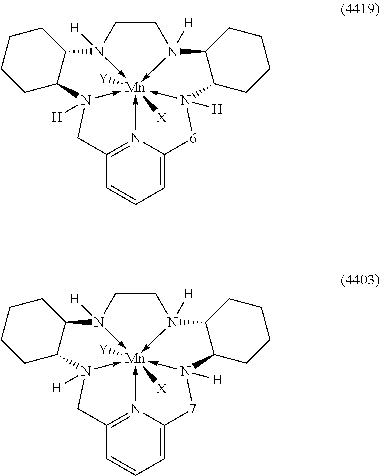

Embodiment 34. The process according to any preceding Embodiment, wherein the pentaaza macrocyclic ring complex corresponding to the compound of formula (I)(a) or (I)(b) is any selected from at least one of the complexes below:

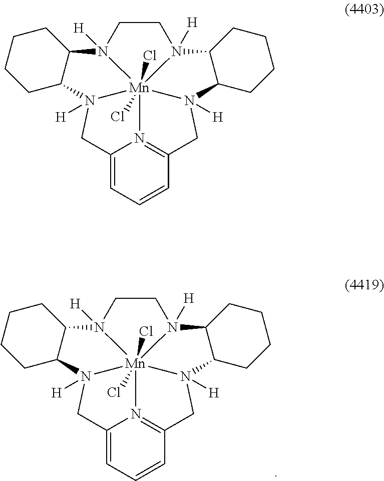

Embodiment 35. The process according to any preceding Embodiment, wherein the pentaaza macrocyclic ring complex corresponding to the compound of formula (I)(a) or (I)(b) is any selected from at least one of the complexes below:

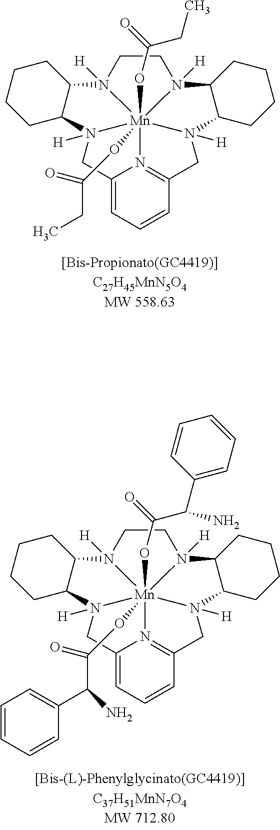

Embodiment 36. The process according to any preceding Embodiment, wherein the cyclization stage is performed in the presence of any one or more of 1-propanol, 2-propanol, ethanol, dimethyl formamide (DMF) and tetrahydrofuran (THF).

Embodiment 37. The process according to any preceding Embodiment, wherein the cyclization stage is performed in the presence of 1-propanol.

Embodiment 38. The process according to any preceding Embodiment, wherein the cyclization stage is performed in the presence of a tertiary amine base having a pKa above about 9, the tertiary amine base comprising the formula NR₁R₂R₃, wherein R₁, R₂ and R₃ comprise cyclic or non-cyclic alkyl groups, with at least one of R₁, R₂ and R₃ being a non-cyclic alkyl group, and at least one of R₁, R₂ and R₃ having 3 carbon atoms or less.

Embodiment 39. The process according to any preceding Embodiment, wherein the tertiary amine base comprises a pKa above about 10.

Embodiment 40. The process according to any preceding Embodiment, wherein each of R₁, R₂ and R₃ are non-cyclic alkyl groups.

Embodiment 41. The process according to any preceding Embodiment, wherein the tertiary amine base comprises DIPEA.

Embodiment 42. The process according to any preceding Embodiment, wherein the catalytic reduction reaction is performed in the presence of a catalyst comprising palladium on carbon, palladium black, PtO₂, Raney Ni 2800, activated carbon, palladium on Al₂O₃, platinum on carbon, iridium on carbon, ruthenium on carbon, rhodium on carbon, sponge nickel catalyst, and palladium on BaSO₄.

Embodiment 43. The process according to any preceding Embodiment, wherein the catalytic reduction reaction is performed in the presence of a catalyst comprising palladium on carbon.

Embodiment 44. The process according to any preceding Embodiment, wherein the catalytic reduction reaction is performed in the presence of a catalyst comprising from 5 wt % to 20 wt % palladium on carbon.

Embodiment 45. The process according to any preceding Embodiment, wherein the catalytic reduction reaction is performed in the presented of catalyst comprising from 8 wt % to 15 wt % palladium on carbon.

Embodiment 46. The process according to any preceding Embodiment, wherein the catalytic reduction reaction is performed in the presence of a catalyst comprising about 10 wt % palladium on carbon.

Embodiment 47. The process according to any preceding Embodiment, wherein the catalytic reduction reaction is performed in the presence of from about 1.25% w/w to about 10% w/w (on an anhydrous basis) catalyst.

Embodiment 48. The process according to any preceding Embodiment, wherein the catalytic reduction reaction is performed in the presence of from about 2.5% w/w to about 5% w/w (on an anhydrous basis) catalyst.

Embodiment 49. The process according to any preceding Embodiment, wherein the catalytic reduction reaction is performed in the presence of about 2.5% w/w (on an anhydrous basis) catalyst.

Embodiment 50. The process according to any preceding Embodiment, wherein the catalyst comprises from 1 wt % to 60 wt % of water.

Embodiment 51. The process according to any preceding Embodiment, wherein the catalyst comprises from 1.5% wt % to 55 wt % of water.

Embodiment 52. The process according to any preceding Embodiment, wherein the catalyst comprises from 30 wt % to 55 wt % of water.

Embodiment 53. The process according to any preceding Embodiment, wherein the catalyst comprises about 50 wt % of water.

Embodiment 54. The process according to any preceding Embodiment, wherein the cyclization stage is performed at a temperature in the range of 50° C. to 130° C.

Embodiment 55. The process according to any preceding Embodiment, wherein the catalytic reduction reaction is performed at a temperature in the range of from 50° C. to 120° C.

Embodiment 56. The process according to any preceding Embodiment, wherein the catalytic reduction reaction is performed at a temperature in the range of from 50° C. to 100° C.

Embodiment 57. The process according to any preceding Embodiment, wherein the catalytic reduction reaction is performed at a temperature in the range of from 70° C. to 90° C.

Embodiment 58. The process according to any preceding Embodiment, wherein the catalytic reduction reaction is performed at a temperature of about 85° C.

Embodiment 59. The process according to any preceding Embodiment, wherein the catalytic reduction reaction is performed in the presence of about 25 to about 500 psi hydrogen.

Embodiment 60. The process according to any preceding Embodiment, wherein the catalytic reduction reaction is performed in the presence of about 25 to about 100 psi hydrogen.

Embodiment 61. The process according to any preceding Embodiment, wherein the catalytic reduction reaction is performed in the presence of about 30 to about 100 psi hydrogen.

Embodiment 62. The process according to any preceding Embodiment, wherein the catalytic reduction reaction is performed in the presence of about 50 psi hydrogen.

Embodiment 63. The process according to any preceding Embodiment, wherein the catalytic reduction reaction is performed in the presence of propanol.

Embodiment 64. A process of preparing a bisimine complex of Formula (II)(a) or (II)(b) below:

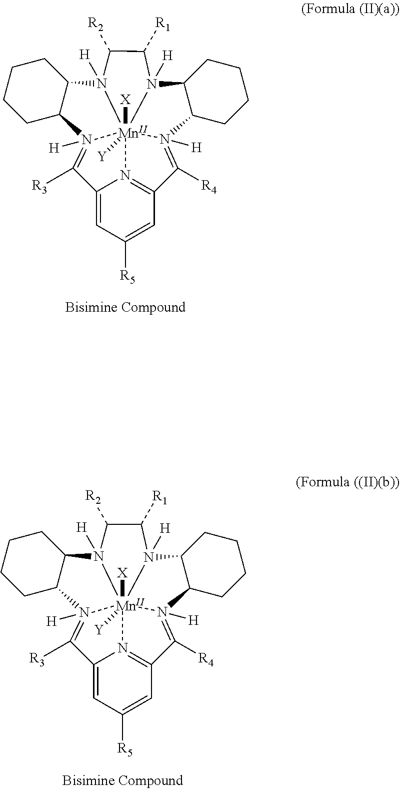

wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
the process comprising, in a cyclization stage,
(A) reacting a tetraamine product comprising a tetraamine compound of Formula (III)(a)(i) or Formula (III)(a)(ii) below, or a salt thereof, with a diacetyl pyridine compound of Formula (III)(b) below, and a source of manganese (II) ion corresponding to the formula Mn(X)(Y), in the presence of a tertiary amine base, wherein the tetraamine product comprises at least 99% optical purity of the compound of Formula (III)(a)(i) or Formula (III)(a)(ii), to provide a cyclization product comprising a bisimine compound of Formula (II)(a) or (II)(b) having at least 95% optical purity,
wherein the source of manganese (II) ion comprises one or more counter-ions corresponding to X and Y in the bisimine of Formula (II)(a) or Formula (II)(b), and the one or more counter-ions include a counter-ion that is other than halo

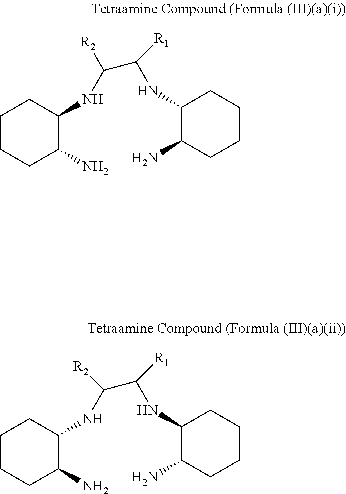

wherein R₁-R₄ are independently selected from group consisting of hydrogen and substituted or unsubstituted alkyl

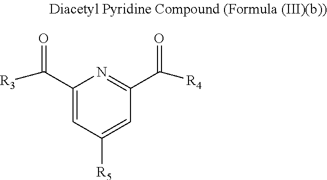

wherein R₃ and R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂

Embodiment 65. The process according to Embodiment 64, wherein the bisimine product comprising the bisimine compound of Formula (II)(a) or (II)(b) has at least 97%, at least 98% and/or at least 99% optical purity.

Embodiment 66. The process according to any preceding Embodiment, wherein the source of manganese (II) ion comprises a counter-ion that is any selected from the group consisting of oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

Embodiment 67. The process according to any preceding Embodiment, wherein the source of manganese (II) ion comprises any of Mn(II) propionate, Mn(II) acetate, and Mn(II) nitrate.

Embodiment 68. The process according to any preceding Embodiment, wherein the source of manganese (II) ion comprises manganese (II) propionate.

Embodiment 69. The process according to any preceding Embodiment, wherein the cyclization stage comprises a process as performed according to any of Embodiments 1-63.

Embodiment 70. The process according to any preceding Embodiment, further comprising performing a reduction stage according to any of Embodiments 1-63.

Embodiment 71. A process of preparing a bisimine complex of Formula (II)(a)(2) or (II)(b)(2) below:

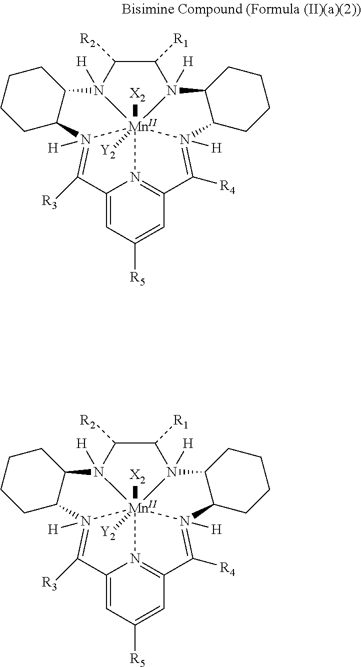

### Bisimine Compound (Formula (II)(b)(2))

wherein X₂ and Y₂ are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
the process comprising reacting a bisimine compound of Formula (II)(a)(1) or (II)(b)(1) below having first ligands X₁ and Y₁, with a source of second ligands X₂ and Y₂ that are other than the first ligands, to provide the bisimine compound of Formula (II)(a)(2) or (II)(b)(2) where X₂ and Y₂ comprise the second ligands that are other than that of the first ligands

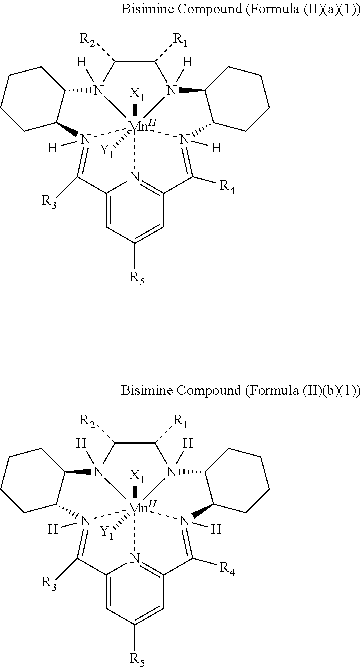

Embodiment 72. The process according to any preceding Embodiment, wherein the first ligands X₁ and Y₁ comprise any recited in Embodiments 1-66, and the second ligands X₂ and Y₂ comprise any recited in Embodiments 1-67 that are different than X₁ and Y₁ and that are other than halo.

Embodiment 73. The process according to any preceding Embodiment, wherein the source of second ligands X₂ and Y₂ comprises any selected from the group consisting of oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

Embodiment 74. The process according to any preceding Embodiment, wherein the source of second ligands X₂ and Y₂ comprises any selected from the group consisting of alkyl carboxylates, aryl carboxylates and arylalkyl carboxylates.

Embodiment 75. The process according to any preceding Embodiment, wherein the first ligands X₁ and Y₁ are chloro.

Embodiment 76. The process according to any preceding Embodiment, wherein the source of second ligands X₂ and Y₂ comprises a source of propionato ligands.

Embodiment 77. The process according to any preceding Embodiment, wherein the source of the second ligands X₂ and Y₂ comprises a salt form of the ligands.

Embodiment 78. The process according to any preceding Embodiment, wherein the source of the second ligands X₂ and Y₂ comprises sodium propionate.

Embodiment 79. The process according to any preceding Embodiment further comprising forming a cyclization product comprising the bisimine compound of Formula (II)(a)(1) or (II)(b)(1) by performing a cyclization stage according to any of Embodiments 1-63 with X and Y in the source of manganese (II) ion corresponding to X₁ and Y₁, prior to reacting the bisimine product comprising the bisimine compound of Formula (II)(a)(1) and (II)(b)(1) with the source of second ligand X₂ and Y₂.

Embodiment 80. The process according to Embodiment 79, wherein the bisimine product provided by the cyclization stage and comprising the bisimine compound of Formula (II)(a)(1) or (II)(b)(1) has at least 95%, at least 97%, at least 98% and/or at least 99% optical purity.

Embodiment 81. The process according to any preceding Embodiment, further comprising performing a reduction stage to reduce the Bisimine Compound of Formula (II)(a)(2) or (II)(b)(2) according to any of Embodiments 1-63.

Embodiment 82. A pentaaza macrocyclic ring complex product comprising a pentaaza macrocyclic ring complex of either Formula (I)(a) or (I)(b) below:

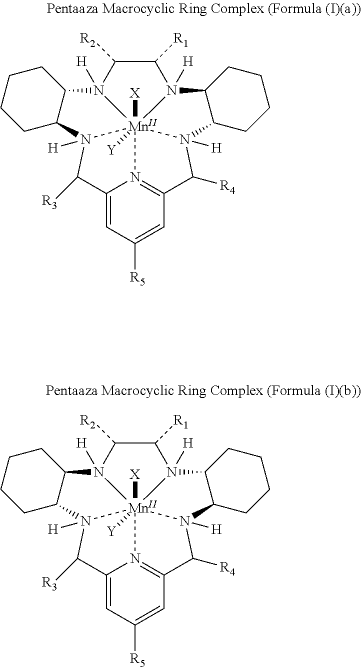

wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
wherein the pentaaza macrocyclic ring complex product comprising the pentaaza macrocyclic ring complex of Formula (I)(a) or (I)(b) is prepared according to the process of any of Embodiments 1-63.

Embodiment 83. The pentaaza macrocyclic ring complex product according to Embodiment 82, wherein the pentaaza macrocyclic ring complex product is not subjected to any optical purification processes after the reduction stage according to the process of any of Embodiments 1-63 is performed.

Embodiment 84. A pentaaza macrocyclic ring complex product comprising the pentaaza macrocyclic ring complex of Formula (I)(a) or Formula (I)(b) of any of Embodiments 82-83, wherein the product comprises the pentaaza macrocyclic ring complex of Formula (I)(a) or (I)(b) having at least 99.0%, at least 99.5%, at least 99.7% and/or at least 99.8% optical purity.

Embodiment 85. A pharmaceutical composition comprising the pentaaza macrocyclic ring complex product of any of Embodiments 82-83.

Embodiment 86. A bisimine complex product comprising a bisimine complex of Formula (II)(a) or (II)(b) below:

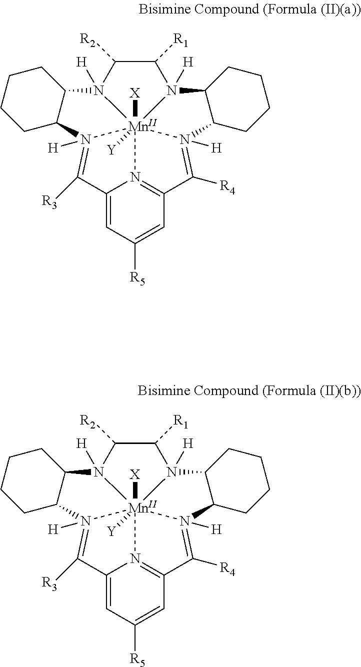

wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
wherein the bisimine complex of Formula (II)(a) or (II)(b) is prepared according to the process of any of Embodiments 64-70.

Embodiment 87. A bisimine complex product comprising a bisimine complex of Formula (II)(a)(2) or (II)(b)(2) below:

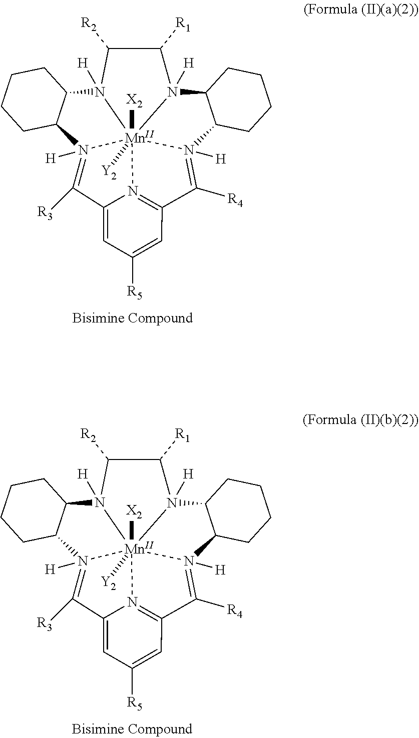

wherein X₂ and Y₂ are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
wherein the bisimine complex of Formula (II)(a) or (II)(b) is prepared according to the process of any of Embodiments 71-81.

Embodiment 88. A bisimine product comprising the bisimine complex of either the compound of Formula (II)(a) or (II)(b) according to any of Embodiments 86-87, wherein the bisimine product comprising the compound of Formula (II)(a) or (II)(b) has at least 97%, at least 98% and/or at least 99% optical purity.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments have been discussed, the above specification is illustrative, and not restrictive. Many variations will become apparent to those skilled in the art upon review of this specification. The full scope of the embodiments should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

1. A process of preparing a pentaaza macrocyclic ring complex of Formula (I)(a) or (I)(b) below: 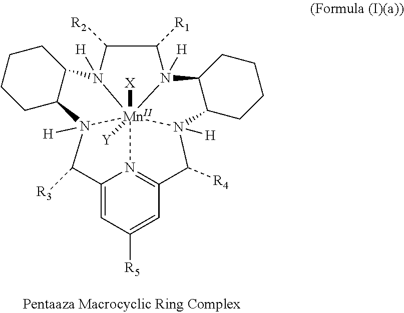 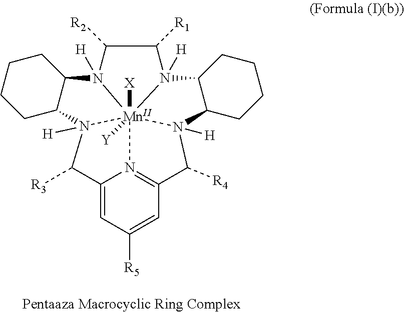
wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
the process comprising,
(A) in a cyclization stage, reacting a tetraamine product comprising a tetraamine compound of Formula (III)(a)(i) or Formula (III)(a)(ii) below, or a salt thereof, with a diacetyl pyridine compound of Formula (III)(b) below, and a source of manganese (II) ion corresponding to the formula Mn(X)(Y), in the presence of a tertiary amine base, wherein the tetraamine product comprises at least 99% optical purity of the compound of Formula (III)(a)(i) or Formula (III)(a)(ii) or salt thereof, 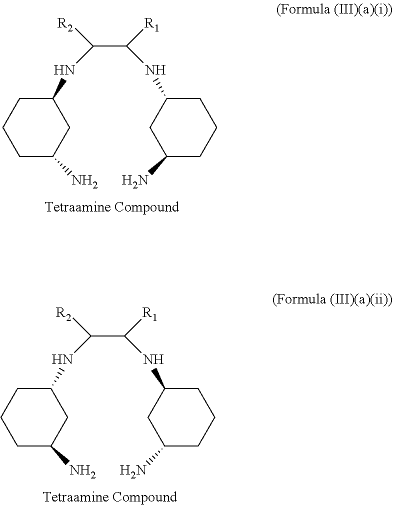
wherein R₁-R₄ are independently selected from group consisting of hydrogen and substituted or unsubstituted alkyl 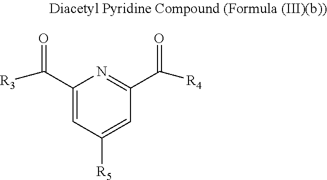
wherein R₃ and R₄ are independently selected from group consisting of hydrogen and substituted or unsubstituted alkyl, and
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂,
to provide a cyclization stage product comprising a bisimine compound of Formula (II)(a) or (II)(b) below having at least 95% optical purity 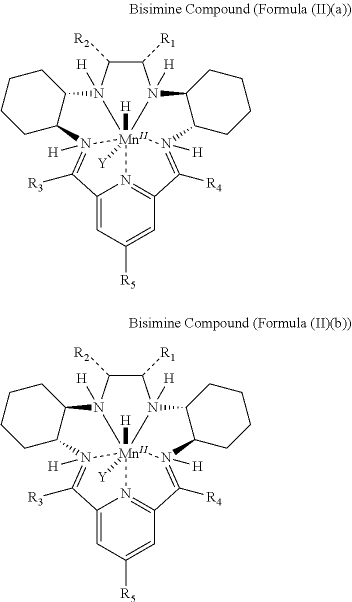
wherein X and Y are independently neutral or negatively charged ligands,
R₁-R₄ are independently selected from group consisting of hydrogen and substituted or unsubstituted alkyl,
R₅ is selected from the group consisting of hydrogen, fluoro, chloro, bromo, iodo, substituted or unsubstituted alkyl, substituted or unsubstituted aryl, substituted or unsubstituted heteroaryl, substituted or unsubstituted cycloalkyl, substituted or unsubstituted heterocycle, substituted or unsubstituted amino, substituted or unsubstituted alkoxy, substituted or unsubstituted alkanolamino, substituted or unsubstituted sulfide, O(CH₂)₂NHCH₃, O(CH₂)₂NH₂, ethoxy, methoxy, methyl, ethyl, butyl, pentyl, phenyl, tertbutyl, benzoyl, O(CH₂)₃CH₃, CN, OH, S(CH₂)₃OH, S(CH₂)₂NH₂, SCH₂(C═O)OCH₃, SCH₂(C═O)OH, S(CH₂)₂O(C═O)CHCH₂, S(CH-₂)₂N(CH₂CH₃)₂, NH₂ and NO₂, and
(B) in a reduction stage performed after at least 50% by weight of the diacetyl pyridine compound of Formula (III)(b) has reacted in the cyclization stage, performing a catalytic hydrogenation reduction reaction on the bisimine compound of Formula (II)(a) or Formula (II)(b) to form a reduction stage product comprising the pentaaza macrocyclic ring complex of Formula (I)(a) or Formula (I)(b)having at least 99% optical purity.

2. The process according to claim 1, wherein the bisimine product provided by the cyclization stage has the bisimine compound of Formula (II)(a) or (II)(b) having at least 97%, at least 98% and/or at least 99% optical purity.

3. The process according to claim 1, wherein the reduction stage product has the pentaaza macrocyclic ring complex of Formula (I)(a) or Formula (I)(b) having least 99.5%, at least 99.7% and/or at least 99.8% optical purity.

4. The process according to claim 1, wherein the reduction stage product comprising the pentaaza macrocyclic ring complex of Formula (I)(a) or Formula (I)(b) is not subjected to any further optical purification processes after the reduction stage is performed.

5. The process according to claim 1, wherein the reduction stage is performed after at least 60% by weight, at least 75% by weight, at least 80% by weight, at least 90% by weight and/or at least 95% by weight of the diacetyl pyridine compound of Formula (III) has reacted in the cyclization stage.

6. The process according to claim 1, wherein the reduction stage comprises adding a catalytic hydrogenation reduction catalyst and a source of hydrogen to the bisimine compound of Formula (II)(a) and/or (II)(b), and is performed at least 30 mins, at least 1 hour and/or at least 2 hours after the diacetyl pyridine compound is added in the cyclization stage.

7. The process according to claim 1, further comprising isolating the bisimine compound of Formula (II)(a) or Formula (II)(b) prior to the reduction stage.

8. The process according to claim 1, further comprising removing impurities from the bisimine product of the cyclization stage, prior to performing the reduction stage.

9. The process according to claim 8, comprising removing sulfur-containing impurities.

10. The process according to claim 8, comprising removing dimethyl sulfide impurities.

11. The process according to claim 9, comprising removing impurities from the bisimine product to provide a product having less than 10 ppm, less than 5 ppm, less than 1 ppm, less than 0.5 ppm and/or less than 0.1 ppm of sulfur-containing impurities.

12. The process according to claim 1, wherein X and Y are independently selected from the group consisting of halo, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof in any of Formulas (I)(a), (I)(b), (II)(a) and (II)(b).

13. The process according to claim 1, wherein X and Y independently selected from the group consisting of fluoro, chloro, bromo and iodo anions in any of Formulas (I)(a), (I)(b), (II)(a) and (II)(b).

14. The process according to claim 1, wherein X and Y independently selected from the group consisting of alkyl carboxylates, aryl carboxylates and arylalkyl carboxylates in any of Formulas (I)(a), (I)(b), (II)(a) and (II)(b).

15. The process according to claim 1, wherein X and Y are chloro in any one of Formulas (I)(a), (I)(b), (II)(a) and (II)(b).

16. The process according to claim 1, wherein X and Y are propionato in any one of Formulas (I)(a), (I)(b), (II)(a) and (II)(b).

17. The process according to claim 1, wherein X and Y in any of Formulas (I)(a) and (I)(b) are other than X and Y in any of Formulas (II)(a) and (II)(b).

18. The process according to claim 1, wherein X and Y in any of Formulas (I)(a) and (I)(b) are the same as X and Y in any of Formulas (II)(a) and (II)(b).

19. The process according to claim 1, wherein X and Y in any of Formulas (II)(a) and (II)(b) are each chloro, and X and Y in any of Formulas (I)(a) and (I)(b) are each propionato.

20. The process according to claim 1, wherein the source of manganese (II) ion comprises, as counter-ions to the manganese ion, moieties corresponding to the ligands X and Y in any one of Formulas (I)(a), (I)(b), (II)(a) and (II)(b).

21. The process according to claim 1, wherein the source of manganese (II) ion comprises Mn (II) with counter-ions corresponding to any selected from the group consisting of halo, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

22. The process according to claim 1, wherein the source of manganese (II) ion comprises any of Mn(II) chloride, Mn(II) bromide and Mn(II) iodide.

23. The process according to claim 1, wherein the source of manganese (II) ion comprises any of Mn(II) propionate, Mn(II) acetate, and Mn(II) nitrate.

24. The process according to claim 1, wherein the compound of Formula (II)(a) or (II)(b) comprises X and Y corresponding to a first ligand moiety, and wherein the process further comprises the intermediate step of reacting the compound of Formula (II)(a) or (II)(b) with a source of a second ligand moiety to provide a compound of Formula (II)(a) or (II)(b) having X and Y corresponding to the second ligand moiety, prior to performing the reduction stage (B).

25. The process according to claim 24, wherein the first ligand moiety comprises a chloro ligand, and the second ligand moiety comprises a propionato ligand, and wherein the compound of Formula (I)(a) or (I)(b) comprises propionato ligands for X and Y.

26. The process according to claim 1, wherein the process further comprises reacting the compound of Formula (I)(a) or (I)(b) with a source of another ligand moiety to introduce X and Y corresponding to the other ligand moiety.

27. The process according to claim 26, wherein the process comprises reacting the pentaaza macrocyclic ring complex corresponding to the compound of formula (I)(a) or (I)(b) having X and Y ligands corresponding to chloro, with a source of a propionato ligand, to replace chloro with propionato as the X and Y ligands moiety in the compound of Formula (I)(a) or (I)(b).

28. The process according to claim 1, wherein the pentaaza macrocyclic ring complex corresponding to the compound of formula (I)(a) or (I)(b) is any selected from the group consisting of Formulae (V)-(XVI) below:

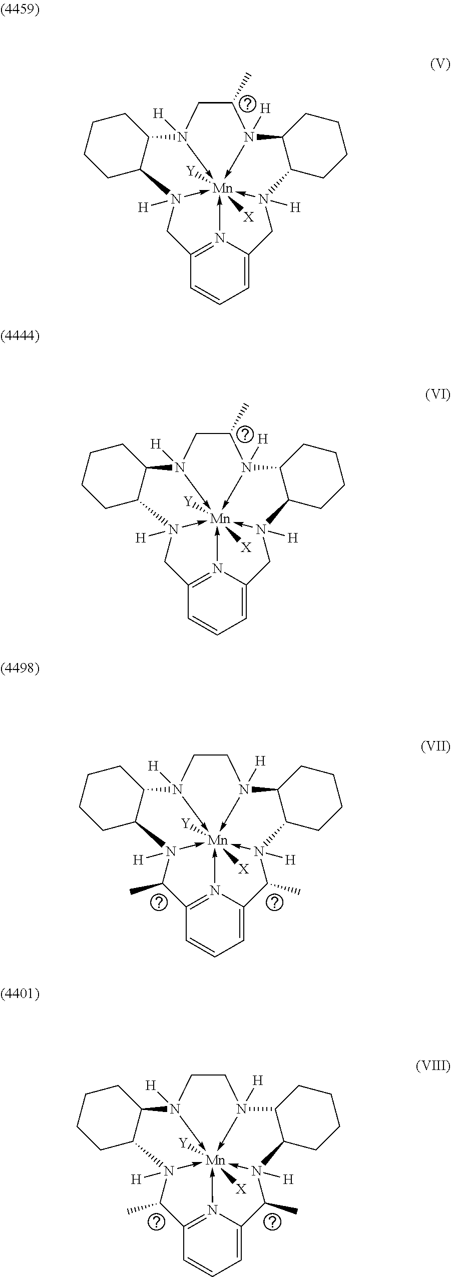

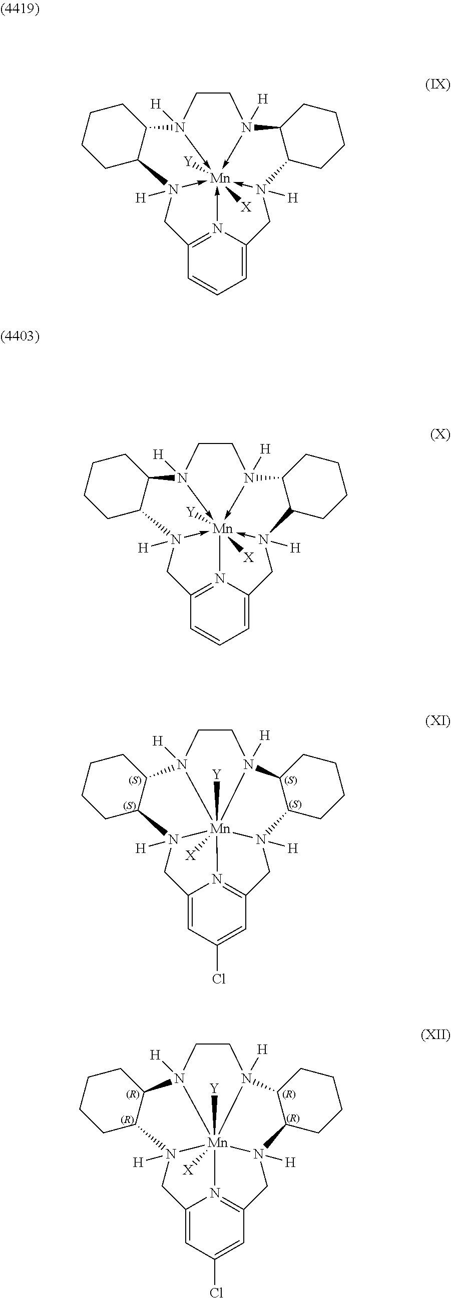

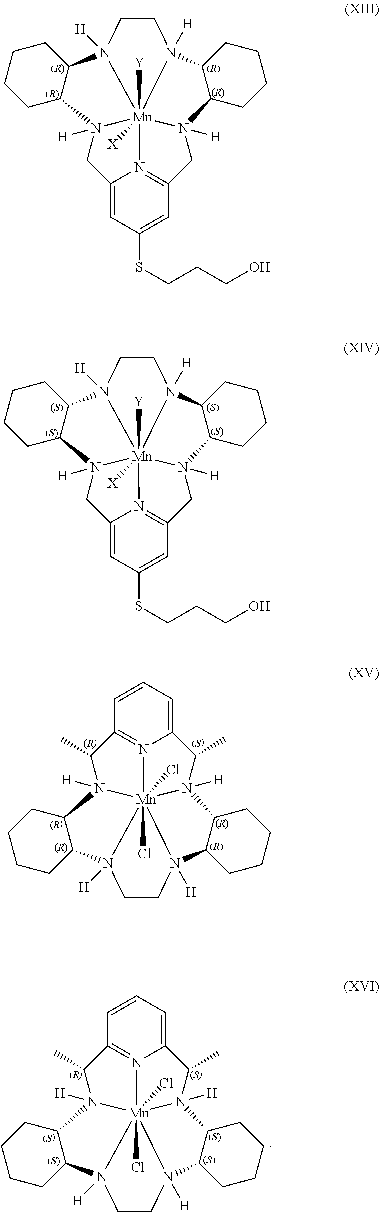

29. The process according to claim 1, wherein the pentaaza macrocyclic ring complex corresponding to the compound of formula (I)(a) or (I)(b) is any selected from the group consisting of Formulae (IE_{R1}), (IE_{S1}), (IE_{R2}), (IE_{S2}), (IE_{R3}), and (IE_{S3}) below: 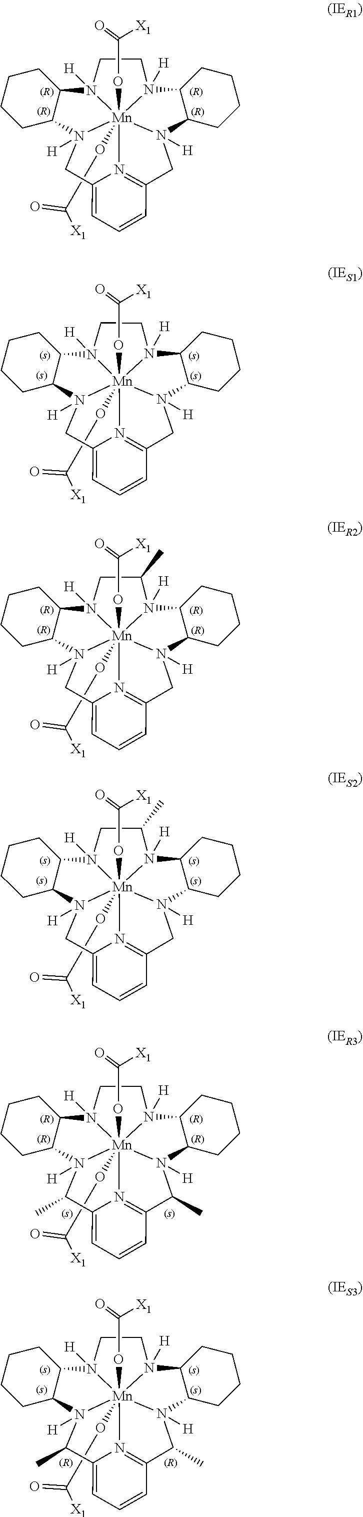
wherein
each X₁ is independently substituted or unsubstituted phenyl or —C(—X₂)(—X₃)(—X₄);
each X₂ is independently substituted or unsubstituted phenyl or alkyl;
each X₃ is independently hydrogen, hydroxyl, alkyl, amino, —X₅C(═O)R₁₃ where X₅ is NH or O, and R₁₃ is C₁-C₁₈ alkyl, substituted or unsubstituted aryl or C₁-C₁₈ aralkyl, or —OR₁₄, where R₁₄ is C₁-C₁₈alkyl, substituted or unsubstituted aryl or C₁-C₁₈ aralkyl, or together with X₄ is (═O);
each X₄ is independently hydrogen or together with X₃ is (═O); and
the bonds between the transition metal M and the macrocyclic nitrogen atoms and the bonds between the transition metal M and the oxygen atoms of the axial ligands —OC(═O)X₁ are coordinate covalent bonds.

30. The process according to claim 29, wherein within any of the Formulae (IE_{R1}), (IE_{S1}), (IE_{R2}), (IE_{S2}), (IE_{R3}), and (IE_{S3}), X₁ is —C(—X₂)(—X₃)(—X₄) and each X₂, X₃, and X₄, in combination, corresponds to any of the combinations identified in the following table:

| Combination | X₂ | X₃ | X₄ |
|---|---|---|---|
| 1 | Ph | H | H |
| 2 | Ph | OH | H |
| 3 | Ph | NH₂ | H |
| | | |
|---|---|---|
| 4 | Ph | ═O |
| | | (X₃ and X₄ in |
| | | combination) |
| | | | |
|---|---|---|---|
| 5 | Ph | CH₃ | H |
| 6 | CH₃ | H | H |
| 7 | CH₃ | OH | H |
| 8 | CH₃ | NH₂ | H |
| | | |
|---|---|---|
| 9 | CH₃ | ═O |
| | | (X₃ and X₄ in |
| | | combination) |

31.-88. (canceled)
